(19) United States
(12) Patent Application Publication
(10) Pub. No.: US 2022/0298121 A1
(43) Pub. Date: Sep. 22, 2022

(54) AMINE TERMINATED PREPOLYMER AND COMPOSITION COMPRISING THE SAME

(71) Applicants: SOPREMA, STRASBOURG (FR); UNIVERSITE DE HAUTE ALSACE, Mulhouse Cédex (FR)

(72) Inventors: Jean-François STUMBE, STRASBOURG (FR); Baptiste CLEMENT, BERMERING (FR); Pascal PICHON, STRASBOURG (FR); Rémi PERRIN, BOERSCH (FR); Pierre Etienne BINDSCHEDLER, STRASBOURG (FR)

(21) Appl. No.: 17/608,914

(22) PCT Filed: May 20, 2020
(86) PCT No.: PCT/EP2020/064165

§ 371 Date: Nov. 4, 2021

(30) Foreign Application Priority Data
May 24, 2019 (EP) 19305659.5

(51) Int. Cl. C07D 241/04; C08G 73/06; C09D 179/04; C09J 179/04; C08L 79/04

(52) U.S. Cl. CPC C07D 241/04; C08G 73/0633; C09D 179/04; C09J 179/04; C08L 79/04

(57) ABSTRACT

The invention relates to an amine terminated prepolymer and to a curable composition containing this prepolymer. These compositions are used to make sealants, coatings or adhesives useful in the field of construction, public works and civil engineering.

### TECHNICAL FIELD

The invention relates to an amine terminated prepolymer and to a curable composition containing said prepolymer. These compositions are used to manufacture sealants, coatings or adhesives useful in the field of construction, public works and civil engineering.

### BACKGROUND OF THE INVENTION

In public works or construction works, it is necessary to protect structures, generally made of concrete, against any infiltration of water. To do this, sealants or coatings are applied on the structures.

The use of liquid compositions is preferred over prefabricated membranes as they are easier to apply and lead to flexible and continuous membranes that adhere to the structure.

Sealants or coatings can be obtained from acrylic dispersions in aqueous solution which harden on loss of water. However, these products have the drawback of hardening at the surface after application, forming a very thin coating which makes the evaporation of water difficult, thus giving rise to the formation of blisters. These products cure slowly, especially in cold weather, they are very sensitive to rain before they have totally cured, and they form blisters in summer. What is more, these products show poor resistance to prolonged immersion in water, and are therefore unsuitable for waterproofing horizontal flat surfaces. Finally, their mechanical strength is insufficient for use on traffic-bearing surfaces.

Sealants or coatings obtained with polyurethane resins are also known, for example two-component compositions or one-component compositions containing significant amounts of solvents and/or plasticizers. Polyurethane resins contain residual diisocyanates which are considered as harmful to health and to the environment since they may release free diisocyanate monomers. Further, the use of solvents generates compositions having the following drawbacks:
an unpleasant odor due to the volatile organic compounds,
a toxicity that results in specific labeling and specific operating conditions,
problems with regard to environmental regulations.

Additionally, the use of inert exogenous plasticizers generates compositions having the following drawbacks:
weakening of the mechanical strength,
weakening of the adhesion,
reduced aging over time,
increased water absorption.

There is still a need for prepolymers and liquid two-component curable compositions to provide elastomeric sealants, coatings or adhesives that exhibit one or more of the following properties:
0-1% by weight of free isocyanate monomers
0-5% by weight of solvent
fast curing at room temperature (20-25° C.)
complying with the requirements of a liquid waterproofing system in terms of elasticity, hydrophobicity, hydrolysis resistance, mechanical properties (tensile strength and elongation) and durability.

### SUMMARY OF THE INVENTION

A first object of the present invention is a prepolymer represented by formula (1):

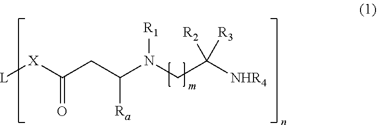

wherein L, X, R₁, R₂, R₃, R₄, Rₐ, m and n are as defined herein.

The invention also aims at providing a method for preparing a prepolymer, wherein said method comprises reacting an electrophile of formula (4) or (Prep) with a secondary diamine of formula (5):

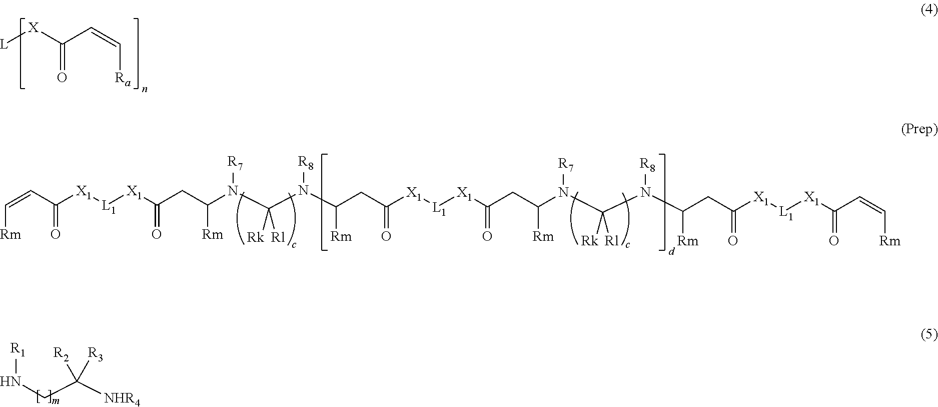

wherein X, X₁, L, L₁, R₁, R₂, R₃, R₄, R₇, R₈, Rₐ, Rₖ, Rₗ, Rₘ, c, d, m and n are as defined herein;

the molar ratio between the hydrogens on the amine reactive groups of the secondary diamine and the α,β-unsaturated carbonyl groups of the electrophile being from 1.50 to 2.20, preferably 1.80 to 2.10, more preferably 1.90 to 2.00.

Another object of the present invention is a composition comprising a prepolymer according to the invention or a mixture thereof; and a multifunctional resin selected from a multifunctional (meth)acrylate resin, a multifunctional epoxy resin, a multifunctional isocyanate resin, a multifunctional carboxylic acid resin, a multifunctional maleimide resin, a multifunctional acrylamide resin, a multifunctional cyclic carbonate resin, an aminoplast resin, and mixtures thereof.

Yet another object of the present invention is a sealant, coating or adhesive obtained by curing the composition according to the invention, preferably at a temperature of −10 to 50° C., in particular −5 to 45° C., more particularly 0 to 40° C., during a time of 1 to 72 h, in particular 2 to 30 h, more particularly 3 to 24 h.

A final object of the present invention is the use of the composition according to the invention for waterproofing exterior or interior traffic-bearing horizontal surfaces, for making flashings, or for renovating roofs.

### Definitions

The term “plurivalent radical” means any group having one or more, for example two (divalent), three (trivalent), four (tetravalent), five (pentavalent) or six (hexavalent), single bonds as points of attachment to other groups.

The term “hydrocarbyl radical” means a radical containing 1 to 500 carbon atoms. The hydrocarbyl radical may be linear or branched, cyclic or acyclic, saturated or unsaturated, aliphatic or aromatic. The hydrocarbyl radical may be interrupted by one or more functional groups selected from ether (—O—), thioether (-S-), disulfide (—S—S—), ester (—C(O)—O—), amide (—C(O)—NH—), carbamate (—NH—C(O)—O—), urea (—NH—C(O)—NH—), dimethylsiloxane (—Si(Me)₂—O—) and mixtures thereof. One or more of the carbon atoms of the hydrocarbyl radical may be replaced by a nitrogen atom or an isocyanurate group having the following formula:

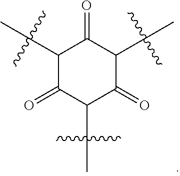 [SMILES: *C1C(=O)C(*)C(=O)C(*)C1=O]

The hydrocarbyl radical may be unsubstituted or substituted by one or more substituents as defined below.

The term “alkyl” means a hydrocarbyl containing 1 to 20 carbon atoms. The alkyl groups may be linear or branched, acyclic or cyclic. Examples include methyl, ethyl, n-propyl, isopropyl, cyclopropyl, n-butyl, isobutyl, sec-butyl, tert-butyl, n-pentyl, cyclopentyl, 2-methylbutyl, 2,2-dimethylpropyl, n-hexyl, cyclohexyl, 2-methylpentyl, 2,2-dimethylbutyl, n-heptyl, 2-methylhexyl, and the like. The term “C1-C20 alkyl” means an alkyl containing 1 to 20 carbon atoms.

When the suffix “ene” or “diyl” is used in conjunction with an alkyl or alkenyl group, this means that the group contains two single bonds as points of attachment to other groups (divalent radical).

The term “aryl” means a polyunsaturated aromatic hydrocarbyl containing one ring (i.e. phenyl), several fused rings (for example naphthyl) or several rings linked via a covalent bond (for example biphenyl), which typically contain 6 to 20, and preferentially 6 to 12, carbon atoms, and wherein at least one ring is aromatic. The aromatic ring may optionally comprise one to two additional fused rings (i.e. cycloalkyl, heterocycloalkyl or heteroaryl). The term “aryl” also encompasses partially hydrogenated derivatives of the carbocyclic system is described above. Examples include phenyl, naphtyl, biphenyl, phenanthrenyl, naphthacenyl, and the like. The term “C6-C12 aryl” means an aryl containing 6 to 12 carbon atoms.

The term “alkylaryl” means a linear or branched alkyl substituent containing a carbon atom attached to an aryl ring. Examples include benzyl, naphthylmethyl, phenethyl, and the like. The term “C6-C12 alkylaryl” means an alkylaryl containing 6 to 12 carbon atoms.

The term “X forms a cycle with Y” means that X and Y, together with the atoms to which they are attached, form an optionally substituted cycle. Examples of cycles are a succinimide, a piperidine, or a piperazine, respectively represented by the following formulae

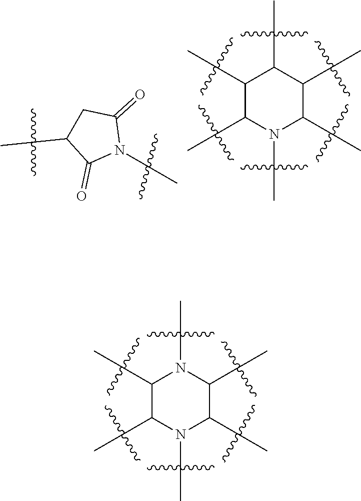 [SMILES: *C1CC(=O)N(*)C1=O ; *C1C(*)C(*)N(*)C(*)C1* ; *C1C(*)N(*)C(*)C(*)N1*]

The following groups: hydrocarbyl radical, alkyl, aryl, alkylaryl and cycle may be unsubstituted or substituted with one or more standard substituents selected from: halogen, alkyl, aryl, hydroxy (—OH), alkoxy (—OR), haloalkyl, cyano (—CN), carboxyl (—COOH), oxo (═O), formyl (—CHO), ester (—COOR), imido (=NR), amido (—CONHR), a tertiary amino group (—NR₂), nitro (—NO₂), sulfonyl (—SO₂—R) wherein each R is independently C1-C20 alkyl, C6-12 aryl or C6-C12 alkylaryl group.

The term “halogen” refers to chlorine, bromine, fluorine and iodine.

The term “haloalkyl” means an alkyl substituted by a halogen atom. Examples include fluoro-, chloro-, bromo-, or iodo-methyl, -ethyl, -propyl, -isopropyl, -butyl, -isobutyl, -tert-butyl, and the like.

The term “haloaryl” means an aryl substituted by a halogen atom.

The term “haloalkylaryl” means an alkylaryl substituted by a halogen atom.

The term “alkoxy” means a —OR group, where R represents an alkyl, cycloalkyl, aryl or alkylaryl group. Examples include methoxy, ethoxy, propoxy, butoxy, cyclohexyloxy, phenoxy, benzyloxy, and the like.

The term “hydrocarbyl radical derived from an alkane” means a hydrocarbyl radical obtained by removing one or more terminal hydrogens from an alkane. Said radical may further be functionalized as defined above.

The term “hydrocarbyl radical derived from a polyether” means a hydrocarbyl radical interrupted by one or more ether functional groups (—O—). Said radical may further be functionalized as defined above.

The term “hydrocarbyl radical derived from a polyester” means a hydrocarbyl radical interrupted by one or more ester functional groups (—C(O)—). Said radical may further be functionalized as defined above.

The term “hydrocarbyl radical derived from a polydimethyl siloxane” means a hydrocarbyl radical interrupted by one or more dimethylsiloxane functional groups (—Si(Me)₂—O—). Said radical may further be functionalized as defined above.

The term “hydrocarbyl radical derived from poly(alkyl (meth)acrylate)” means a hydrocarbyl radical substituted by one or more ester functional groups (—COO(C1-C20 alkyl)). Said radical may further be functionalized as defined above.

The term “hydrocarbyl radical derived from a polybutadiene” means a hydrocarbyl radical comprising one or more butenediyl monomeric units. Said radical may further be functionalized as defined above.

The term “hydrocarbyl radical derived from a polysulfide” means a hydrocarbyl radical interrupted by one or more thioether functional groups (—S—). Said radical may further be functionalized as defined above.

The term “hydrocarbyl radical derived from a polyurethane” means a hydrocarbyl radical interrupted by one or more urethane functional groups (—NH—C(O)—O—). Said radical may further be functionalized as defined above.

The term “hydrocarbyl radical derived from an epoxy acrylate” means a hydrocarbyl radical comprising a moiety obtained by reacting an multifunctional epoxy resin and an acrylic acid. Said radical may further be functionalized as defined above.

The term “multifunctional (meth)acrylate resin” means a compound or polymer comprising at least two (meth)acrylate groups.

The term “multifunctional epoxy resin” means a compound or polymer comprising at least two epoxy groups.

The term “multifunctional isocyanate resin” means a compound or polymer comprising at least two isocyanate groups.

The term “multifunctional carboxylic acid resin” means a compound or polymer comprising at least two carboxylic acid groups.

The term “multifunctional maleimide resin” means a compound or polymer comprising at least two maleimide groups.

The term “multifunctional acrylamide resin” means a compound or polymer comprising at least two acrylamide groups.

The term “multifunctional cyclic carbonate resin” means a compound or polymer comprising at least cyclic carbonate groups.

The term “aminoplast resin” means a compound or polymer formed via the condensation of formaldehyde with an optionally substituted melamine or an optionally substituted urea, comprising at least two hydroxymethyl groups.

The term “liquid composition” means that the composition flows under its own weight. In particular, a liquid composition may exhibit a viscosity between 1,000 and 40,000 centipoises, said viscosity being measured at 23° C. using a Brookfield viscometer (for viscosities of less than 10,000 centipoises, the measurements are taken with the R5 module at a speed of 30 rpm and for viscosities of greater than 10,000 centipoises, the measurements are taken with the R6 module at a speed of 20 rpm). Such a viscosity allows the application of the composition especially with a roller commonly known as a fabric roller or a brush to form 0.5 to 2 mm thick layers in one or more applications.

The term “two-component composition” means a composition comprising two components that are mixed together before application. The composition is applied in a limited time span (a few hours) after being mixed.

The term “curable composition” means a composition comprising a polymer having functional groups capable of forming covalent bonds with chain extenders, cross-linkers or other polymer molecules to form a cross-linked polymer network.

The term “non-toxic composition” means a composition that contains less than 1% by weight of free diisocyanate monomers, according to directive 67/548/EEC (30th ATP directive 2008/58/EC), the free diisocyanate monomer content being measured by gas chromatography coupled to a mass spectrometer (according to standard EN ISO 17734-1/2006).

The term “solvent” means any solvent that is conventionally used in curable compositions, said solvent being inert toward the reagents contained in the composition, liquid at room temperature and having a boiling point below 240° C.

### DETAILED DESCRIPTION OF THE INVENTION

### Prepolymer

The prepolymer of the invention is represented by formula (1):

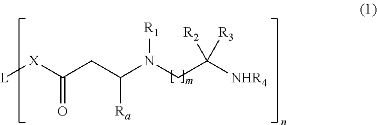

wherein

X is O or NR_{b}, preferably X is O;

L is a plurivalent radical;

Rₐ is hydrogen;

R_{b} is hydrogen;

or Rₐ forms a cycle with R_{b}, preferably a succinimide;

R₁ and R₄ are independently C1-C20 alkyl, C6-C12 aryl or C6-C12 alkylaryl, preferably methyl, ethyl, phenyl or benzyl, more preferably methyl;

or R₁ and R₄ form a cycle, preferably a piperazine, more preferably a piperazine optionally substituted with one or more groups selected from C1-C20 alkyl, C6-C12 aryl C6-C12 alkylaryl, halogen, C1-C20 haloalkyl, C6-C12 haloaryl and C6-C12 haloalkylaryl, even more preferably a piperazine optionally substituted with one or more groups selected from methyl, ethyl, chloro, phenyl, chlorophenyl and benzyl, more preferably still substituted with methyl;

R₂ and R₃ are independently selected from H, C1-C20 alkyl, C6-C12 aryl, C6-C12 alkylaryl, halogen, C1-C20 haloalkyl, C6-C12 haloaryl or C6-C12 haloalkylaryl, preferably H, methyl, ethyl, chloro, phenyl, chlorophenyl or benzyl, more preferably H or methyl, with the proviso that at least one of R₂ and R₃ is not H;

2≤n≤6, preferably 2≤n≤4, more preferably 2≤n≤3;

m is 1, 2, 3, 4, 5 or 6, preferably m is 1.

In particular, groups X and Ra may be selected to form a moiety selected from propanoate, propanamide, and succinimide. As such, the prepolymer of the invention may be represented by one of the following formula (1a)-(1c):

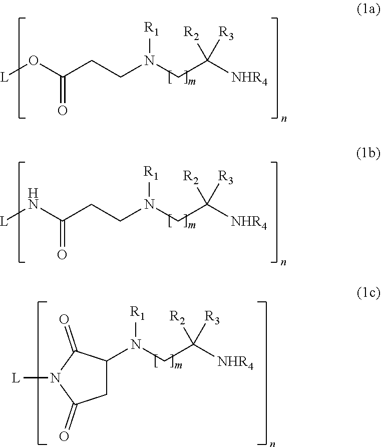

wherein L, R₁, R₂, R₃, R₄, m and n are as defined above.

In a preferred embodiment, the prepolymer of the invention is represented by formula (1a).

The prepolymer of the invention may comprise a piperazine moiety or an ethylenediamine moiety. As such, the following moiety (2) present in formula (1) of the prepolymer

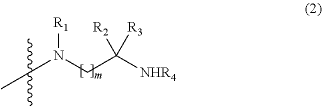

may be represented one of the following formulae (2a)-(2b):

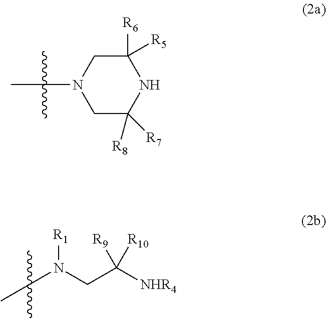

wherein

R₅, R₆, R₇ and R₈ are independently H, C1-C20 alkyl, C6-C12 aryl, C6-C12 alkylaryl, halogen, C1-C20 haloalkyl, C6-C12 haloaryl or C6-C12 haloalkylaryl, preferably H, methyl, ethyl, chloro, phenyl, chlorophenyl or benzyl, more preferably H or methyl; with the proviso that at least one of R₅, R₆, R₇ and R₈ is not H;

R₉ and R₁₀ are independently H, C1-C20 alkyl, C6-C12 aryl, C6-C12 alkylaryl, halogen, C1-C20 haloalkyl, C6-C12 haloaryl or C6-C12 haloalkylaryl, preferably H, methyl, ethyl, chloro, phenyl, chlorophenyl or benzyl, more preferably H or methyl; with the proviso that at least one of R₉ and R₁₀ is not H;

R₁ and R₄ are independently C1-C20 alkyl, C6-C12 aryl or C6-C12 alkylaryl, preferably methyl, ethyl, phenyl or benzyl, more preferably methyl.

In a preferred embodiment, the moiety of formula (2) is represented by formula (2a) wherein R₅ is C1-C20 alkyl, R₇ is H or C1-C20 alkyl, and R₆ and R₈ are H. In a particularly preferred embodiment, the moiety of formula (2) is represented by formula (2a) wherein R₅ is methyl, R₇ is H or methyl, and R₆ and R₈ are H.

In a particularly preferred embodiment, the prepolymer of formula (1) may be represented by the following formula (1′):

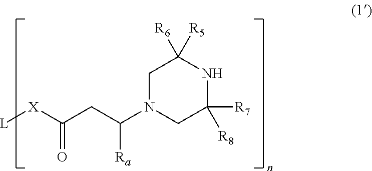

wherein L, X, Rₐ, R₅, R₆, R₇ R₈ and n are as defined above;

even more particularly the prepolymer of formula (1) may be represented by one of the following formulae(1′a)-(1′c):

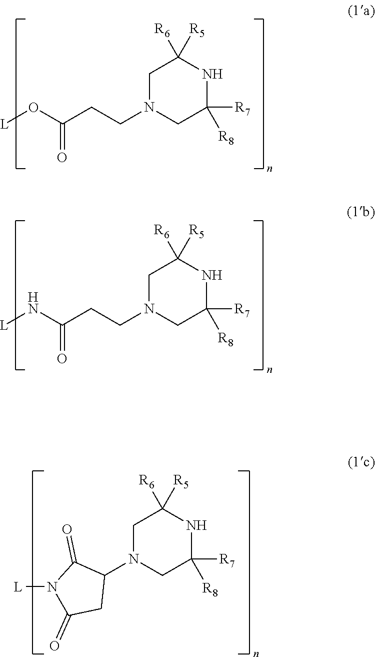

wherein L, R₅, R₆, R₇ R₈ and n are as defined above.

Group L can be any group. In particular, L may be a plurivalent hydrocarbyl radical containing 1 to 500 carbon atoms. Said plurivalent hydrocarbyl radical may be linear or branched, cyclic or acyclic, saturated or unsaturated, aliphatic or aromatic. Said plurivalent hydrocarbyl radical may be interrupted by one or more functional groups selected from ether, thioether, disulfide, ester, amide, carbamate, urea, dimethylsiloxane and mixtures thereof. One or more of the carbon atoms of said plurivalent hydrocarbyl radical may be replaced by a nitrogen atom or an isocyanurate group. Said plurivalent hydrocarbyl radical may be substituted by one or more substituents selected from halogen, alkyl, aryl, hydroxy, alkoxy, haloalkyl, cyano, carboxyl, oxo, formyl, ester, imido, amido, a tertiary amino group, nitro, sulfonyl and mixtures thereof.

In particular, L may be a plurivalent hydrocarbyl radical derived from: an alkane;

a polyether, preferably a polypropylene glycol, a copolymer of ethylene glycol and propylene glycol or a polytetramethylene glycol;

a polyester, preferably a polyester based on a fatty acid dimer;

a polyurethane;

an isocyanurate;

an epoxy acrylate, preferably a bio-based acrylated epoxidized resin;

a polydimethyl siloxane;

a poly(alkyl (meth)acrylate);

a polybutadiene;

a polysulfide;

and combinations thereof.

Preferably, L is a plurivalent hydrocarbyl radical derived from an alkane, a polyether, a polyurethane and combinations thereof. More preferably, L is a plurivalent hydrocarbyl radical comprising 3 to 200 carbon atoms derived from an alkane, a polyether, a polyurethane and combinations thereof. Even more preferably, L is a plurivalent hydrocarbyl radical comprising 100-150 carbon atoms derived from a polyether, a plurivalent hydrocarbyl radical comprising 3-20 carbon atoms derived from an alkane or a plurivalent hydrocarbyl radical comprising 100-150 carbon atoms derived from a polyurethane.

In one embodiment, L may preferably be represented by one of the following formulae (La)-(LI3):

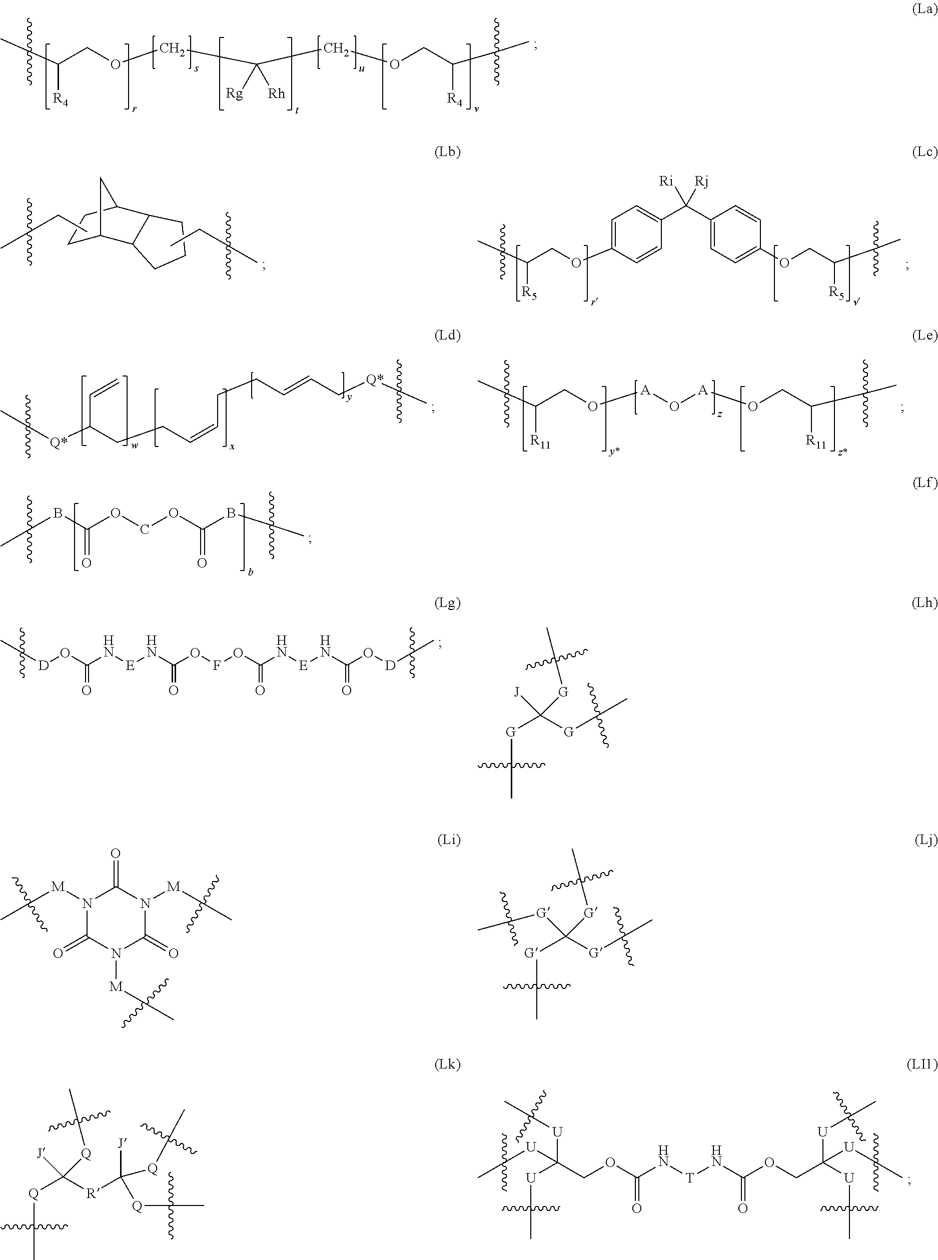

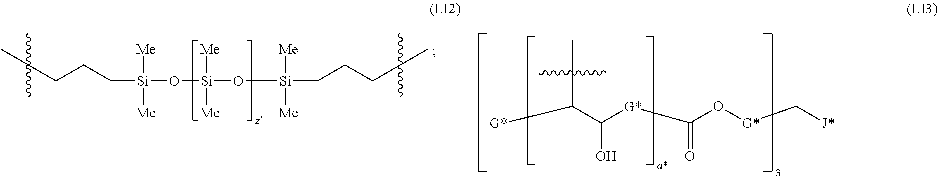

wherein

R_{g} and Rₕ are independently H or C1-C20 alkyl, preferably H, methyl or ethyl, more preferably H or methyl;

Rᵢ and Rⱼ are independently H, halogen, C1-C20 alkyl, C1-C20 haloalkyl, C6-C12 aryl or C6-C12 alkylaryl;

preferably C1-C20 alkyl, more preferably methyl;

each R₄, R₅ and R₁₁ is independently H or methyl;

each A is independently a linear or branched, cyclic or acyclic, saturated or unsaturated alkylene comprising 2 to 20 carbon atoms;

each B is independently a linear or branched, cyclic or acyclic, saturated or unsaturated alkylene comprising 2 to 20 carbon atoms;

C is a linear or branched, cyclic or acyclic, saturated or unsaturated alkylene comprising 4 to 100 carbon atoms optionally interrupted by one or more ether and/or ester functional groups;

each D is independently a linear or branched, cyclic or acyclic, saturated or unsaturated alkylene comprising 2 to 20 carbon atoms;

each E is independently a linear or branched, cyclic or acyclic, saturated or unsaturated alkylene comprising 4 to 100 carbon atoms optionally interrupted by one or more ether and/or carbamate functional groups;

each F is independently a linear or branched, cyclic or acyclic, saturated or unsaturated alkylene comprising 4 to 100 carbon atoms optionally interrupted by one or more ether and/or carbamate functional groups;

each G is independently a linear or branched alkylene comprising 0 to 100 carbon atoms optionally interrupted by one or more ether and/or ester functional groups;

each G′ is independently a linear or branched alkylene comprising 0 to 100 carbon atoms optionally interrupted by one or more ether and/or ester functional groups;

each G* is independently a linear or branched alkylene comprising 0 to 100 carbon atoms J, J′ and J* are independently H or a linear or branched alkyl comprising 1 to 20 carbon atoms, optionally substituted by hydroxy or alkoxy;

each M is independently a linear or branched, cyclic or acyclic alkylene comprising 1 to 20 carbon atoms optionally interrupted by one or more ether and/or carbamate functional groups;

each Q is independently a linear or branched alkylene comprising 0 to 100 carbon atoms optionally interrupted by one or more ether functional groups;

each Q* is independently a linear or branched alkylene comprising 0 to 100 carbon atoms optionally interrupted by one or more ether and/or ester functional groups;

R′ is a linear or branched alkylene comprising 1 to 20 carbon atoms optionally interrupted by one or more ether functional groups;

T is a linear or branched, cyclic or acyclic, saturated or unsaturated alkylene comprising 4 to 100 carbon atoms optionally interrupted by one or more ether and/or carbamate functional groups;

each U is independently a linear or branched alkylene comprising 0 to 100 carbon atoms optionally interrupted by one or more ether and/or ester functional groups;

b is 1 to 10;

s, t and u are independently 0 to 10;

r, r′, v, v′, w, x, y, y*, z and z* are independently 0 to 50;

z′ is 5 to 150

each a* is independently 1, 2 or 3 with the proviso that that formula (LI3) does not comprise more than six a* units.

Preferably, L may be represented by one of formula (La), (Lb), (Lg) or (Lh).

In a preferred embodiment, L may be represented by one of the following formulae (Lb), (Ld) and (Lm)-(Ly):

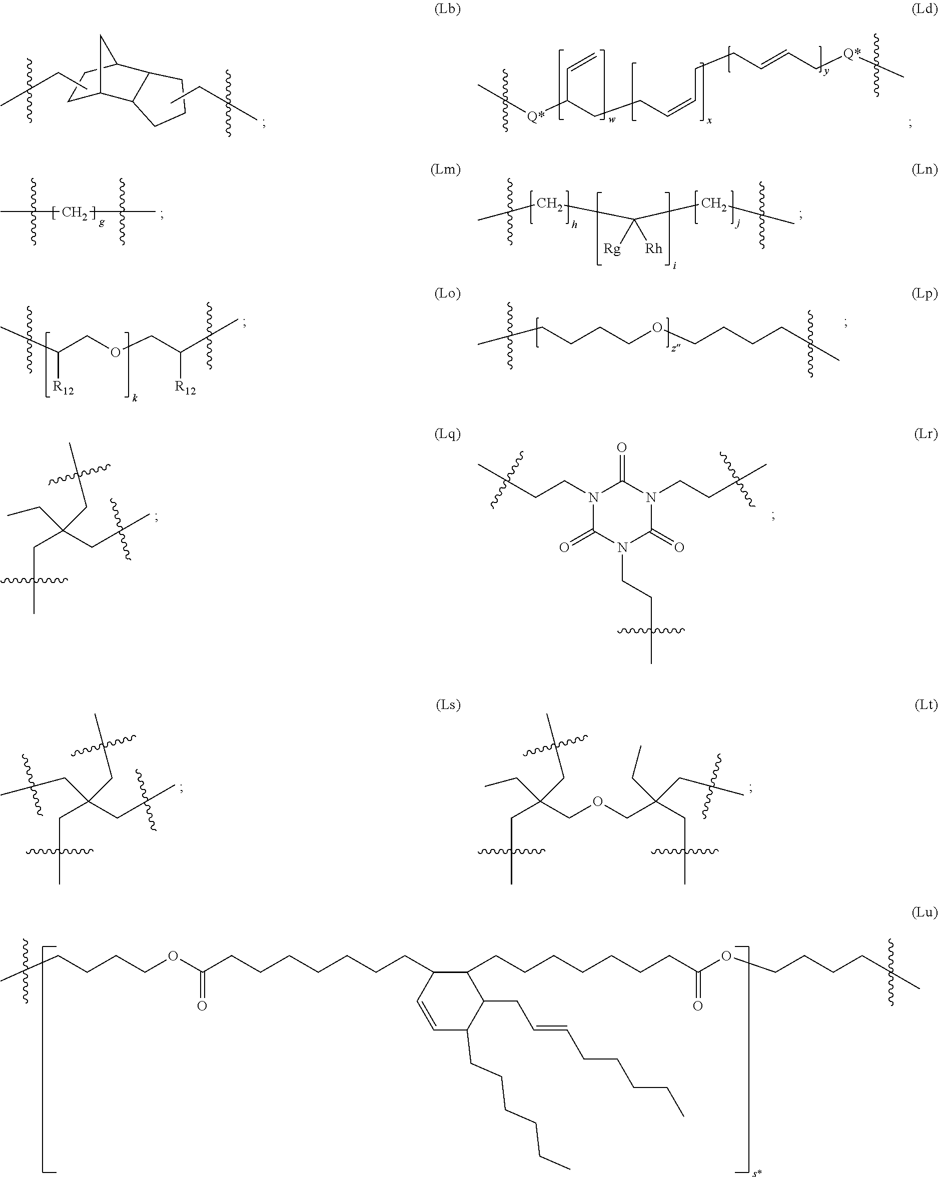

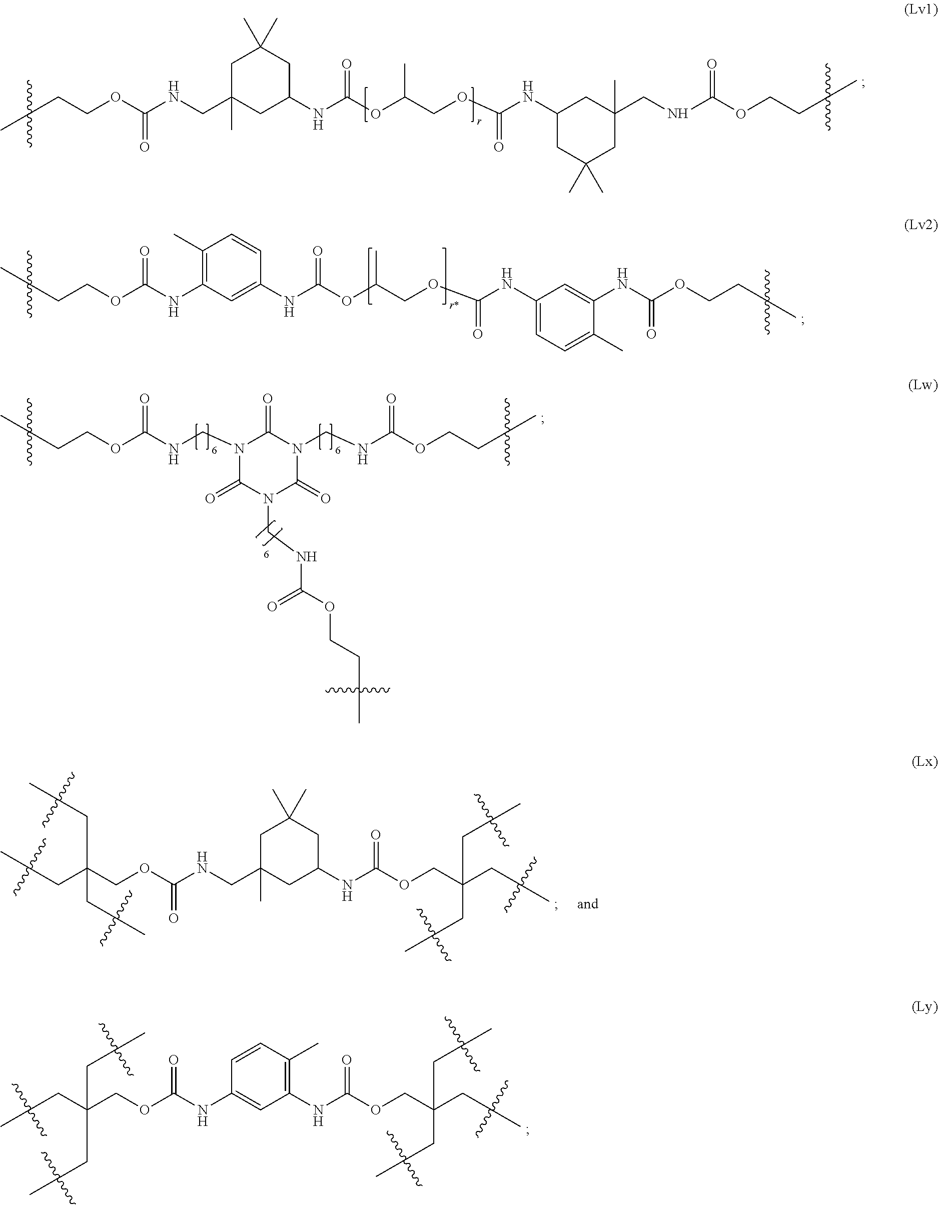

wherein

Q*, w, x and y are as defined above;

each R₁₂ is independently H or methyl;

R_{g} and Rh are independently H or C1-C20 alkyl, preferably H, methyl or ethyl, more preferably H or methyl;

g is 2 to 20, preferably 3 to 12, more preferably 4 to 10;

h, i and j are independently 0 to 10, preferably 1 to 4, more preferably 1 to 2;

k is 2 to 100;

r and r* are independently 1 to 70;

s* is 1 to 20;

z″ is 5 to 50, preferably 8 to 30, more preferably 10 to 20.

Preferably, L may be represented by one of formulae (Lb), (Lm), (Lq) and (Lv2).

In another embodiment, L may be represented by the following formula (LpreP):

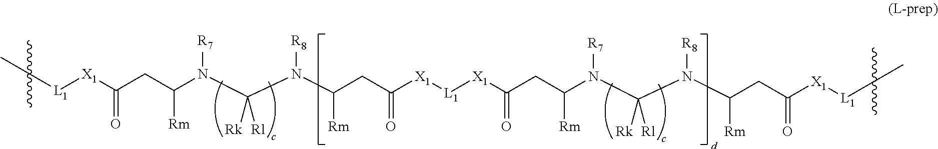

wherein

X₁ is O or NRₙ, preferably X, is O;

each L₁ is independently a plurivalent radical, preferably each L₁ having a molecular weight above 500 g·mol⁻¹;

Rₖ is hydrogen;

Rₗ is hydrogen;

or one Rₗ forms a cycle with R₇, another Rₗ forms a cycle with R₈ and the remaining Rₗ are hydrogen, preferably one Rₗ forms a piperidine with R₇, another Rₗ forms a piperidine with R₈ and the remaining Rₗ are hydrogen;

Rₘ is hydrogen;

Rₙ is hydrogen;

or Rₘ forms a cycle with Rₙ, preferably a succinimide;

R₇ and R₈ are independently C1-C20 alkyl, C6-C12 aryl or C6-C12 alkylaryl, preferably methyl, ethyl, phenyl or benzyl, more preferably methyl;

or R₇ and R₈ form a cycle, preferably a piperazine, more preferably a non-substituted piperazine;

or R₇ forms a cycle with one Rₗ and R₈ forms a cycle with another Rₗ, preferably R₇ forms a piperidine with one Rₗ and R₈ forms a piperidine with another Rₗ;

c is 2, 3, 4, 5, 6, 7, 8 or 9;

0<d≤20, preferably 0.5≤d≤10, more preferably 1≤d≤6, even more preferably 1≤d≤4.

Preferably X, corresponds to X as defined above for the prepolymer and each L₁ corresponds to L as defined above for the prepolymer.

The prepolymer of the invention may exhibit a number average molecular weight (Mn) of 400 to 10,000, preferably 800 to 6,000, more preferably 1,000 to 5,000. The number average molecular weight may be determined by steric exclusion chromatography (SEC) or nuclear magnetic resonance (NMR).

The prepolymer of the invention may be obtained according to the method described below.

### Method for Preparing the Prepolymer of the Invention

The prepolymer of the invention may be obtained by a Michael addition. Michael addition is a chemical reaction in which an enolate anion (nucleophile) reacts with an activated α,β-unsaturated carbonyl compound (electrophile) according to a 1,4-addition. A wide range of functional groups possess sufficient nucleophilicity to react in a Michael addition, such as amines (aza-addition) and thiols (thio-addition). Michael addition is one of the most versatile reactions in organic synthesis with its click chemistry nature, no byproducts, and the mild conditions required for the reaction. An example of a Michael addition is represented in the scheme below:

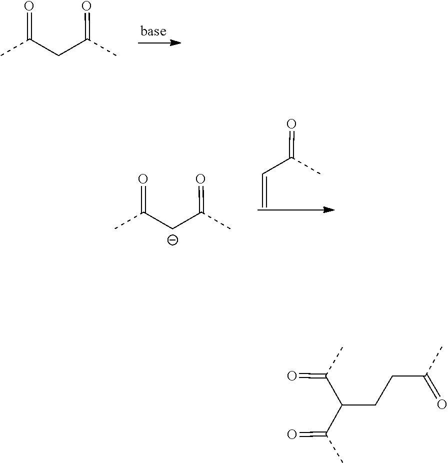

The first step of a Michael reaction is transforming a ketone to an enolate, or nucleophile, through deprotonation due to the addition of a base. This negative charge initiates 1,4-addition on an α,β-unsaturated carbonyl compound which is then protonated and forms the final product. The reaction is thermodynamically controlled as the donors are active methylenes and the acceptors are activated olefins.

In accordance with an aspect, a Michael addition reaction can be employed to manufacture amine terminated polymers useful for obtaining two-component curable sealants, coatings or adhesives. The method involves reacting a multifunctional α,β-unsaturated carbonyl compound with a secondary diamine. The secondary diamine is a Michael donor and the multifunctional α,β-unsaturated carbonyl compound is a Michael acceptor.

The method for preparing a prepolymer according to the invention comprises reacting an electrophile of formula (4) or (Prep) with a secondary diamine of formula (5):

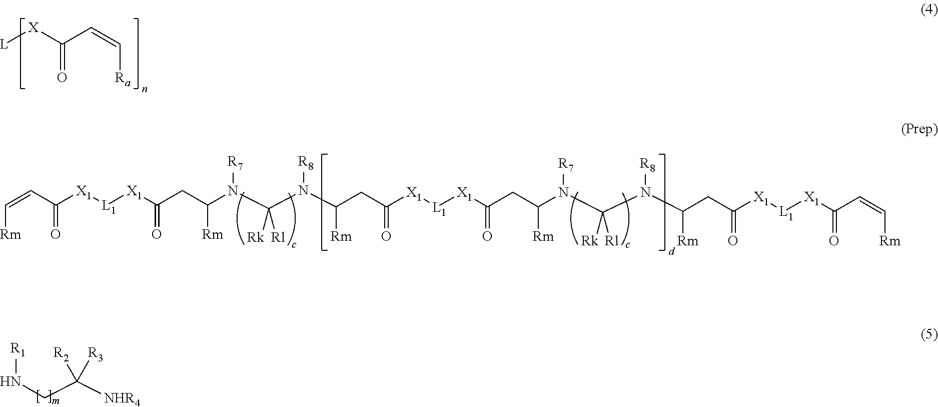

wherein X, X₁, L, L₁, R₁, R₂, R₃, R₄, R₇, R₈, Rₐ, Rₖ, Rₗ, Rₘ, c, d, m and n are as defined above for the prepolymer;

the molar ratio between the hydrogens on the amine reactive groups of the secondary diamine and the α,β-unsaturated carbonyl groups of the electrophile being from 1.50 to 2.20, preferably 1.80 to 2.10, more preferably 1.90 to 2.00.

In the method of the invention, the secondary diamine may react with a mixture of electrophiles, for example a mixture of electrophiles of formula (4) and/or (Prep).

In one embodiment, the electrophile may be represented by one of the following formulae (4a)-(4c) or (PrepA)-(PrepC):

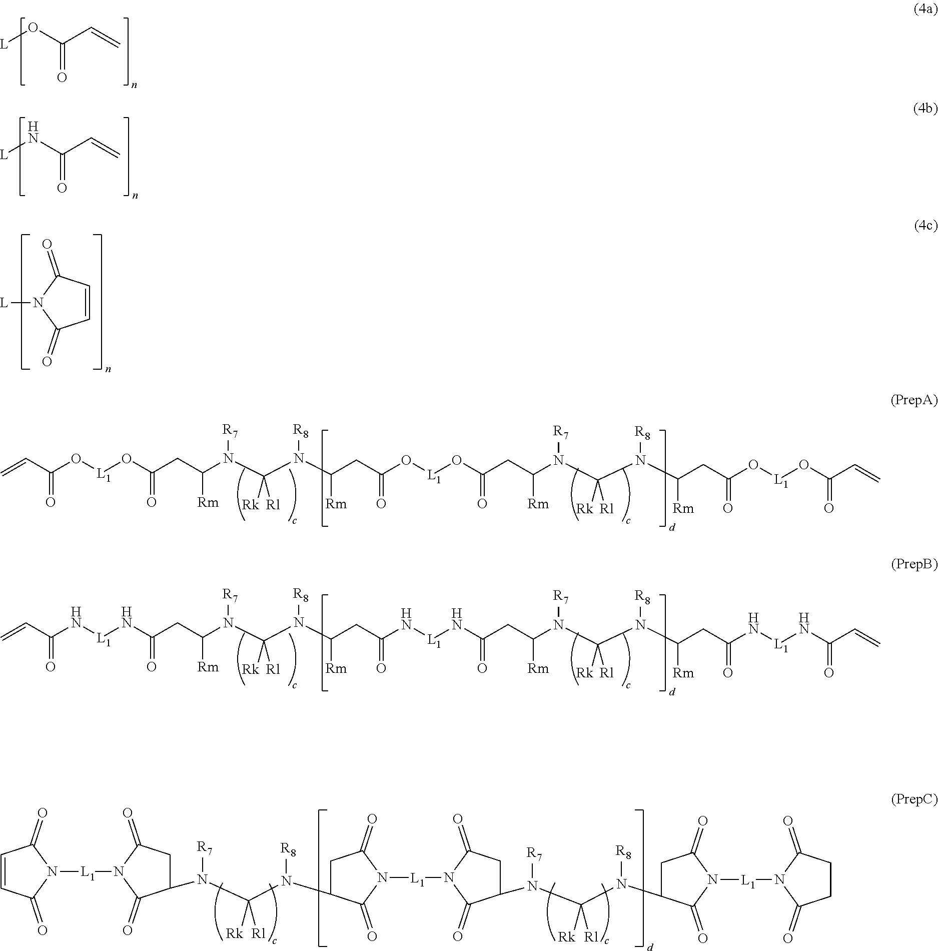

wherein L, L₁, Rₖ, Rₗ, R₇, R₈, c, d and n are as defined above for the prepolymer.

In a preferred embodiment, the electrophile may be represented by formula (4a) or (PrepA).

Preferably, in formulae (4a)-(4c), L may be represented by one of formulae (La)-(LI3) as defined above for the prepolymer, more preferably L may be represented by one of formulae (La), (Lb), (Lg) or (Lh), even more preferably L may be represented by one of formulae (Lb), (Ld) and (Lm)-(Ly), more preferably still L may be represented by one of formulae (Lb), (Lm), (Lq) and (Lv2).

Examples of electrophiles of formula (4a) include a poly(propylene glycol) diacrylate, a poly(ethylene glycol) diacrylate, butanediol diacrylate, 1,6-hexanediol diacrylate, an ethoxylated 1,6-hexanediol diacrylate, 1,10-decanediol diacrylate, 3-methyl-1,5-pentanediol diacrylate, neopentylglycol diacrylate, a propoxylated neopentylglycol diacrylate, dimethylol tricyclodecane diacrylate, an ethoxylated bisphenol A diacrylate, trimethylol propane triacrylate, an ethoxylated trimethylol propane triacrylate, a propoxylated trimethylol propane triacrylate, tris[2-(acryloyloxy)ethyl] isocyanurate, pentaerythritol triacrylate, glycerol triacrylate, a propoxylated glycerol triacrylate, pentaerythritol tetracrylate, an ethoxylated pentaerythritol tetracrylate, an epoxydized soybean oil (AESO) and a polycaprolactone triacrylate.

Another example of an electrophile of formula (4a) is an esterdiol diacrylate (available under reference SR 606A by Sartomer) having the following formula:

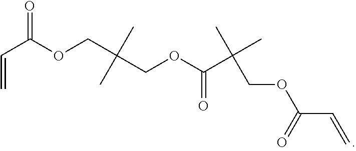 [SMILES: C=CC(=O)OCC(C)(C)COC(=O)C(C)(C)COC(=O)C=C]

Another example of an electrophile of formula (4a) is an aliphatic urethane acrylate oligomer (available under reference CN 9002 by Sartomer) having the following formula:

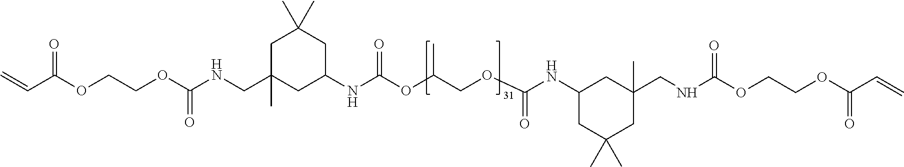

or an aromatic urethane oligomer having the following formula:

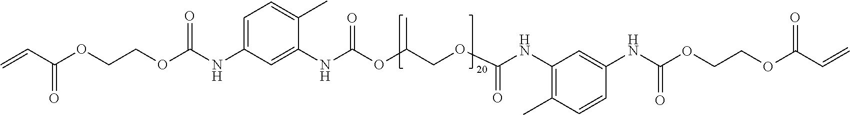

Another example of an electrophile of formula (4a) is a polybutadiene diacrylate (available under reference SR 307 by Sartomer) having the following formula:

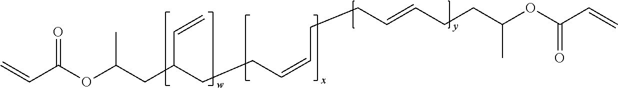

wherein w+x+y=40.

In a particularly preferred embodiment, the electrophile is selected from 1,6-hexanediol diacrylate, dimethylol tricyclodecane diacrylate, trimethylol propane triacrylate, an aromatic urethane oligomer and mixtures thereof.

Electrophiles of formula (Prep) and (PrepA)-(PrepC) are described in patent application number EP19305656.1 filed on May 24, 2019 by the Applicants.

An example of a suitable electrophile of formula (PrepA) is represented below:

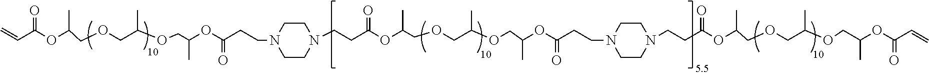

The secondary diamine of formula (5) may be represented by one of the following formulae (5a) or (5b):

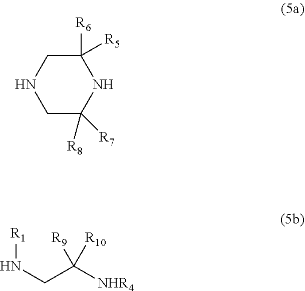

wherein R₁, R₄, R₅, R₆, R₇, R₈, R₉ and R₁₀ are as defined above for the prepolymer.

Preferably the secondary diamine is represented by formula (5a) wherein R₅ is C1-C20 alkyl, R₇ is H or C1-C20 alkyl, and R₆ and R₈ are H. Even more preferably, the secondary diamine is represented by formula (5a) wherein R₅ is methyl, R₇ is H or methyl, and R₆ and R₈ are H.

In the method of the invention, the reaction between the electrophile and the secondary diamine may be carried out in the presence or in the absence of a solvent. Preferably, the reaction between the electrophile and the secondary diamine is carried out in the absence of a solvent.

In the method of the invention, the reaction between the electrophile and secondary diamine may be carried out in the presence or in the absence of a catalyst. In particular, said catalyst may be a base, more particularly 1,4-diazabicyclo[2.2.2]octane (DABCO) or 1,8-diazabicyclo[5.4.0]undec-7-ene (DBU). Preferably, the reaction between the electrophile and the secondary diamine is carried out in the absence of a catalyst.

In the method of the invention, the reaction between the electrophile and the secondary diamine may be carried out ata temperature of 10 to 80° C., in particular 15 to 60° C., more particularly 20 to 40° C., during a time of 5 min to 4 h, in particular 15 min to 2 h, more particularly 30 min to 1 h.

The completion of the reaction may be monitored by Fourier-transform infrared (FT IR) spectroscopy. FT IR spectroscopy works by sending infrared radiation through a chemical sample, where some radiation is absorbed into the sample and some passes through. The radiation that is absorbed is converted to vibrational energy, which produces a unique signal that identifies the compound. During the Michael addition, the carbon-carbon double bond of the electrophile is transformed into a carbon-carbon single bond. Once the FT IR signal of the carbon-carbon double bond disappears, the reaction may be considered as finished. The reaction may alternatively be monitored by Proton Nuclear Magnetic Resonance (¹H-NMR). Once the α,β-unsaturated carbonyl group has reacted, the signals of the ethylenic protons (between 5.8 and 6.5 ppm) are no longer visible and a new signals relative to single bonds CH2—CH2 are present.

### Composition Comprising a Prepolymer

The composition according to the invention comprises the prepolymer of the invention and a multifunctional resin. The composition may further optionally comprise an additive, a catalyst and/or a secondary amine terminated prepolymer.

The prepolymer introduced in the composition of the invention is as defined above. The composition may comprise a mixture of prepolymers according to the invention. The composition may comprise a mixture of a prepolymer according to the invention and an amine terminated prepolymer not according to the present invention (such as a secondary amine terminated prepolymer, an aliphatic amine (such as a linear aliphatic amine, cycloaliphatic amine, arylyl amine, polyetheramine (also known as Jeffamine)), a Mannich base adduct or a polyamine).

Thus, the composition may comprise in addition to the prepolymer according to the invention or a mixture of prepolymers according to the invention and a multifunctional resin, at least one amine terminated prepolymer not according to the present invention. This composition may further comprise an additive and/or a catalyst.

The amount of the prepolymer(s) according to the invention in the composition may be from 5 to 99%, in particular 7 to 95%, more particularly 10 to 90%, by weight based on the weight of the composition.

The multifunctional resin that is introduced in the composition may be selected from selected from a multifunctional (meth)acrylate resin, a multifunctional epoxy resin, a multifunctional isocyanate resin, a multifunctional carboxylic acid resin, a multifunctional maleimide resin, a multifunctional acrylamide resin, a multifunctional cyclic carbonate resin, an aminoplast resin, and mixtures thereof.

Preferably, the multifunctional resin is a multifunctional acrylate resin, more preferably a multifunctional polyurethane acrylate resin, a multifunctional polyester acrylate resin (partially or totally bio-based), a bio-based multifunctional epoxide acrylate resin, a multifunctional polyether acrylate resin, a multifunctional isocyanurate acrylate, a prepolymer of formula (Prep), and mixtures thereof,

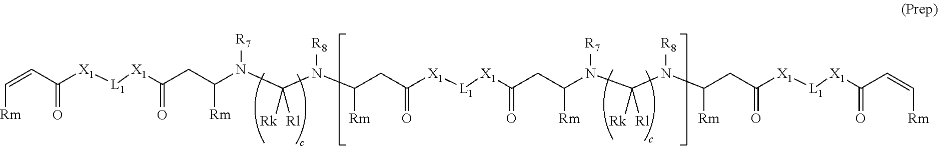

wherein L₁, X₁, Rₖ, Rₗ, Rₘ, R₇, R₈, c and d are as defined above for the prepolymer.

The composition may comprise a mixture of multifunctional resins. In one embodiment, the composition comprises a mixture of multifunctional resins having different functionalities (i.e. a different number of reactive groups). In particular, the composition may comprise a multifunctional resin, preferably a multifunctional acrylate resin, having a functionality higher than 2, preferably from 2.1 to 6, more preferably from 2.5 to 4; and optionally a multifunctional resin, preferably a multifunctional acrylate resin, having a functionality equal to 2. Alternatively, the composition may comprise a multifunctional resin, preferably a multifunctional acrylate resin, having a functionality equal to 2 and optionally a multifunctional resin, preferably a multifunctional acrylate resin, having a functionality higher than 2, preferably from 2.1 to 6, more preferably from 2.5 to 4.

More particularly, the composition may comprise a trifunctional acrylate resin and optionally a difunctional acrylate resin. Alternatively, the composition may comprise a difunctional acrylate resin and optionally a trifunctional acrylate resin.

Even more particularly, the composition may comprise trimethylolpropane triacrylate and optionally tricyclo[5.2.1.0]decanedimethanol diacrylate or 1,6-hexanediol diacrylate. Alternatively, the composition may comprise an aromatic polyurethane diacrylate.

The multifunctional resin introduced in the composition of the invention may exhibit a number average molecular weight of 100 to 10 000, preferably 150 to 4 000, more preferably 200 to 800.

In one embodiment, the molar ratio between hydrogens on the amine reactive groups of the prepolymer(s) (prepolymers according to the invention and prepolymers not according to the present invention, if present) and the reactive groups of the multifunctional resin is from 0.80 to 1.20, preferably 0.90 to 1.10, more preferably 0.95 to 1.05.

The amount of multifunctional resin in the composition may be from 1 to 95%, in particular 5 to 90%, more particularly 10 to 85%, by weight based on the weight of the composition.

The composition may comprise an additive. The additive that is optionally introduced in the composition of the invention is a conventional additive used in the manufacture of sealants, coatings and adhesives. The composition may comprise a mixture of additives. The additive introduced in the composition of the invention is selected from a plasticizer, a filler, an adhesion promoter, a pigment or dye, a UV-absorber, an antioxidant, a UV-stabilizer, a moisture scavenger, a fungicide, a biocide, a root-penetration preventer, a fire-retardant, a rheology modifier, an oxygen barrier and mixtures thereof.

Examples of suitable plasticizers are aromatic oils, such as diisopropyl naphthalene (Ruetasolv® DI) or NYTEX® 820; esters of polycarboxylic acids with linear or branched aliphatic alcohols, such as phthalates and adipates, for example dioctyl phthalate (DOP), diisodecyl phthalate (DIDP), diisononyl phthalate (DINP), butylbenzyl phthalate and di(2-ethylhexyl)adipate (DEHA); esters of polyols with linear or branched carboxylic acids, such as trimethyl pentanediol diisobutyrate (TXIB); alkylsulfonic acid phenylesters, such as Mesamoll®; and mixtures thereof.

Examples of suitable fillers are mineral or organic fillers, such as calcium carbonate, silica, talc, dolomite, kaolin, carbon black, titanium dioxide, and mixtures thereof. Preferably, said filler is calcium carbonate. Fillers derived from recycling can also be used (lignin, recycled fibers, ground polymer materials, coke, ground cement materials).

Examples of suitable biocides and fungicides are 2-octyl-2H-isothiazol-3-one (OIT) in diisododecylphthalate (Fungitrol® PA10), N-(Trichloromethylthio) phthalimide (Fungitrol® 11), 3-iodo-2-propynyl butylcarbamate (IPBC) (Fungitrol® C450 or Preventol® MP100).

An example of a suitable root-penetration preventer is 2-(4-chloro-2-methylphenoxy)-propionic acid octyl ester (Preventol® B5).

Examples of suitable UV-absorbers and antioxidants are Irganox® 565 (2,4-Bis(octylthio)-6-(4-hydroxy-3,5-di-tert-butylanilino)-1,3,5-triazine), IONOL® CP (2,6-Di-tert-butyl-4-methylphenol), Tinuvin® 1130 (2-(2-hydroxyphenyl)-benzotriazole), Tinuvin® 400 (2-hydroxyphenyl-s-triazine).

Examples of suitable UV-stabilizers are Tinuvin® 292 ((Bis(1,2,2,6,6-pentamethyl-4-piperidyl) sebacate), Tinuvin® 123 (Bis(1-octyloxy-2,2,6,6-tetramethyl-4-piperidyl) sebacate).

Examples of suitable moisture scavenger and adhesion promoters are silanes, such as vinyltrimethoxysilane (Geniosil® XL 10) and N-(2-Aminoethyl)-3-aminopropyltrimethoxysilane (Geniosil® GF91).

Examples of suitable rheology modifiers are a hydrophobically modified alkali swellable emulsion (HASE) such as Acrysol® TT 935 and Acrysol® DR-110 ER; a cellulose or cellulose derivative such as CMC, HMC, HPMC; a polysaccharide such as carrageenan, pullulan, konjac, and alginate; a clay such as attapulgite, bentonite and montmorillonite; a gum such as guar gum, xanthan gum, cellulose gum, locust bean gum, and acacia gum.

Examples of suitable fire retardants are borates, such as colemanite, halogenated compounds (tris(chloropropyl)phosphate=TCPP or tetrabromobisphenol-A=TBBA or Hexabromocyclododecane=HBCD), triaryl phosphate, melamine (non-halogenated flame retardant), alumina trihydrate, DOPO (9,10-dihydro-9-oxa-10-phosphaphenanthrene-10-oxide=Polyphlox® 3710).

An example of a suitable oxygen barrier is a wax, such as paraffin wax (Sasolwax® 5603).

The amount of the additive in the composition may be from 0 to 80%, in particular 5 to 60%, more particularly 10 to 40% by weight based on the weight of the composition.

The composition may further comprises a catalyst or a secondary amine terminated prepolymer. The catalyst or a secondary amine terminated prepolymer may be introduced in the composition to accelerate the reaction between the prepolymer(s) and the multifunctional resin. Preferably, the composition does not comprise a catalyst.

The catalyst that may optionally be introduced in the composition may be selected from a tertiary amine, an organometallic compound, an acid, an anhydride, and mixtures thereof. Preferably, the catalyst is a metal carboxylate (tin, zinc, iron, lead, copper or titanium carboxylate such as dibutyltin dilaurate (DBTDL), dioctyltin dilaurate, dioctyltin acetylacetonate, copper acetylacetonate, isopropyl triisostearoyl titanate), a carboxylic or sulfonic acid (stearic acid, palmitic acid, oleic acid, 4-dodecylbenzene sulfonic acid, dinonylnaphthalene disulfonic acid, p-toluenesulfonic acid (p-TSA), methanesulfonic acid), a tertiary cyclic amine (1,8-diazabicyclo[5.4.0]undec-7-ene (DBU), 1,5-Diazabicyclo[4.3.0]non-5-ene (DBN), 1,4-diazabicyclo[2.2.2]octane (DABCO)) or an anhydride (methyltetrahydrophthalic anhydride (MHTPA), methylnadic anhydride and methylsuccinic anhydride). Even more preferably, the catalyst is DBU or DBTDL.

The amount of catalyst in the composition may be from 0 to 2%, in particular 0.01 to 1%, more particularly 0.1 to 0.8%, by weight based on the weight of the composition.

A secondary amine terminated prepolymer not according to the present invention may optionally be introduced in the composition. Such secondary amine terminated prepolymer not according to the present invention may be represented by the following formula (3):

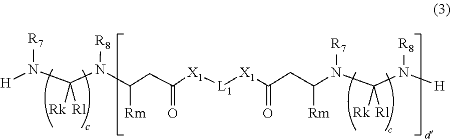

wherein

L₁, X₁, Rₖ, Rₗ, Rₘ, R₇, R₈ and c are as defined above for the prepolymer;

1<d′≤10, preferably 1,5≤d′≤8, more preferably 2≤d′≤6.

Prepolymers of formula (3) are described in patent application number EP19305658.7 filed on May 24, 2019 by the Applicants.

Other examples of amine terminated prepolymer not according to the present invention may optionally be introduced in the composition. Theses amine terminated prepolymer not according to the present invention can be secondary amines or primary amines. Examples of such amine terminated prepolymer not according to the present invention include, but are not limited to, aliphatic amines, Mannich base adducts, and mixtures thereof.

Examples of aliphatic amines include, but are not limited to, linear aliphatic amines, cycloaliphatic amines, arylyl amines, polyetheramines (also known as Jeffamines), and mixtures thereof.

Examples of linear aliphatic amines include, but are not limited to, 2,2,4-trimethylhexamethylenediamine represented by the following formula:

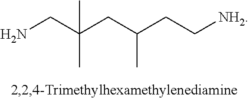

Examples of cycloaliphatic amines include, but are not limited to, IPDA (isophorone diamine), PACM (bis(4-aminocyclohexyl)methane), BMACM (4,4′-Methylenebis(2-methylcyclohexylamine)), 1,2-DACH (1,2-diaminocyclohexane) represented by the following formulae:

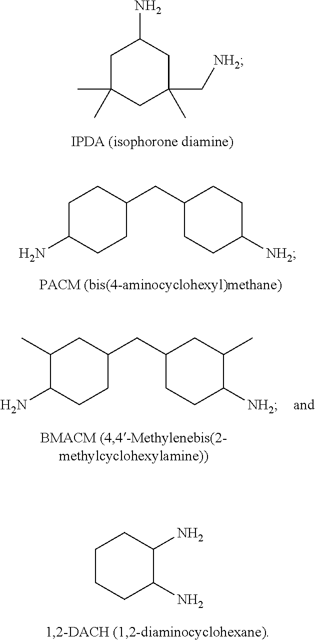 [SMILES: CC1(C)CC(N)CC(C)(CN)C1 ; NC1CCC(CC2CCC(N)CC2)CC1 ; CC1CC(CC2CCC(N)C(C)C2)CCC1N ; "and" ; NC1CCCCC1N]

Examples of arylyl amines include, but are not limited to, 1,3-BAC (1,3-bis[aminoethyle]cyclohexane) and MXDA (meta-xylene diamine) represented by the following formulae:

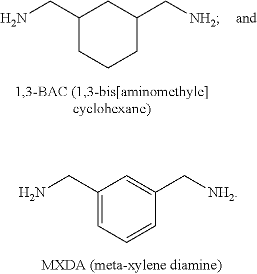 [SMILES: NCC1CCCC(CN)C1 ; "and" ; NCc1cccc(CN)c1]

Examples of polyetheramines (also known as Jeffamines) include, but are not limited to, Jeffamines D (n=[2-70]) and Jeffamines SD (n=[2-70]) represented by the following formulae:

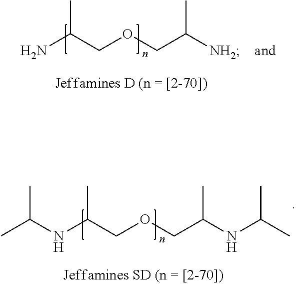

By Mannich base adducts it is understood products resulting from the reaction between a polyamine, a phenolic compound and formaldehyde, and in particular it is understood compounds having the following formula (A):

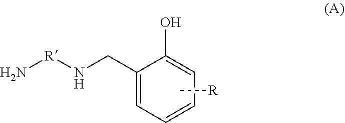

wherein R is H or a saturated or unsaturated C1-C30 alkyl chain and R′ is a polyamine.

In particular, examples of Mannich Base adducts include, but are not limited to, Aradur® 14 commercialized by Hunstman, Epikure® 3251 commercialized by Hexion, phenalkamines and in particular phenalkamines resulting from Mannich reaction between cardanol (Cashew Nut Shell Liquid (CNSL)), formaldehyde and polyamine such as NC-541 commercialized by Cardolite having the following formula:

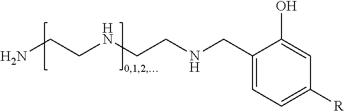

NC-541 (phenalkamine or mannich-based curing agent derived from cashew nut shells) (R=C₁₅H₂₇₋₃₁).

In the specific embodiment wherein the composition according to the present invention further comprises an amine terminated prepolymer not according to the present invention, said amine terminated prepolymer can be a polyamine. Thus, a polyamine not according to the present invention may optionally be introduced in the composition. Such polyamine is for example a priamine, a polyamidoamine, an amidoamines, or a mixture thereof. This composition may further comprise an additive and/or a catalyst.

Examples of polyamine not according to the present invention include, but are not limited to, priamines, polyamidoamines, amidoamines, and mixtures thereof.

Examples of priamines include, but are not limited to, products resulting from dimerisation reaction of fatty acids such as linoleic acids 9,11 and 9,12 by Diels-Alder cycloaddition, followed by reduction (OH=pripol) and then amination to obtain a priamine. The chains of priamine may contain one or more unsaturations obtained by hydrogenation or may contain no unsaturation. Priamine may be represented by the following formula:

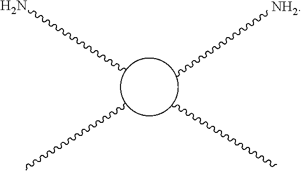

In particular, examples of priamines include, but are not limited to, priamines from the Croda Company such as Priamine® 1071, Priamine® 1073, Priamine® 1074 and Priamine® 1075.

Priamine® 1075 having the following formula is completely unsaturated (equivalent of Versamine® 551 commercialized by BASF):

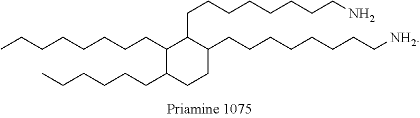 [SMILES: CCCCCCCCC1C(CCCCCC)CCC(CCCCCCCCN)C1CCCCCCCCN]

Priamine® 1074 having the following formula has some unsaturations (equivalent of Versamine® 552 commercialized by BASF:

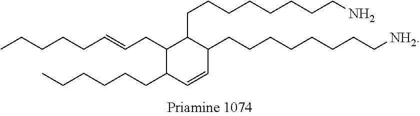 [SMILES: CCCCC/C=C/CC1C(CCCCCC)C=CC(CCCCCCCCN)C1CCCCCCCCN]

Examples of polyamidoamines include, but are not limited to, compounds prepared by the polycondensation reaction between polyamines and dimerized or trimerized fatty acids of vegetable oils where dimerized fatty acids of vegetable oils result from dimerisation reaction of fatty acids such as linoleic acids 9,11 and 9,12 by Diels-Alder cycloaddition. The condensation reaction with polyamines leads to the formation of reactive, amine-terminated polyamides which are polyamidoamines having the following formula (B):

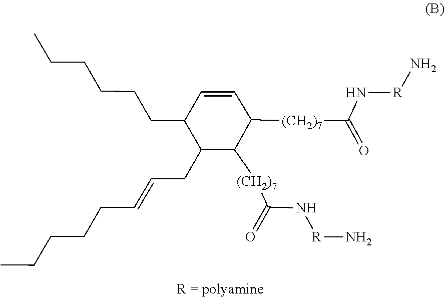

where R represents a polyamine.

In particular, examples of polyamidoamines include, but are not limited to, Versamid® 115 commercialized by BASF having the following formula:

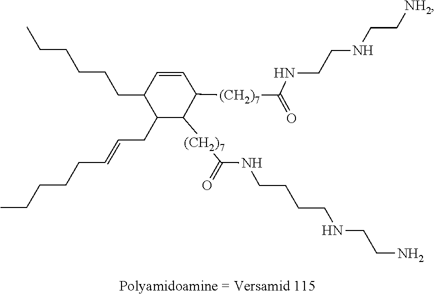

Other examples of polyamidoamines are: Epikure® 3115 commercialized by Hexion and Ancamide® 260 A commercialized by Evonik.

Examples of amidoamines include, but are not limited to, compounds resulting from the reaction between a monoacid with a polyamine such as diethylenetriamine (DETA), leading to a mixture of amidoamines and imidazoline (if cyclization occurs),In particular, examples of amidoamines include, but are not limited to, Epikure® 3010 commercialized by Hexion.

The amount in the composition of amine terminated prepolymer(s) not according to the present invention may be from 0 to 90%, for example from 0 to 70%, in particular 5 to 70%, or 5 to 50%, more particularly 10 to 50% or 10 to 30%, by weight based on the weight of the composition.

For example, the composition of the invention comprises the following constituents, the % being % by weight based on the weight of the composition:

5-99% of the prepolymer of the invention;

1-95% of a multifunctional resin, in particular a trifunctional acrylate resin and/or a difunctional resin;

0-90% of a amine terminated prepolymer;

5-20% of a filler, in particular calcium carbonate;

0-30% of a plasticizer, in particular diisodecyl phthalate (DIDP);

0-2% of a moisture scavenger, in particular vinyltrimethoxysilane;

0-5% of an adhesion promoter, in particular N-(2-Aminoethyl)-3-aminopropyltrimethoxysilane;

0-2% of a catalyst, in particular DBU;

0-10% of a pigment or dye.

In one specific embodiment, the composition of the invention comprises the following constituents, the being % by weight based on the weight of the composition:

5-99% of the prepolymer of the invention;

1-95% of a multifunctional resin, in particular a trifunctional acrylate resin and/or a difunctional resin;

0-90% of a secondary amine terminated prepolymer of formula 3;

5-20% of a filler, in particular calcium carbonate;

0-30% of a plasticizer, in particular diisodecyl phthalate (DIDP);

0-2% of a moisture scavenger, in particular vinyltrimethoxysilane;

0-5% of an adhesion promoter, in particular N-(2-Aminoethyl)-3-aminopropyltrimethoxysilane;

0-2% of a catalyst, in particular DBU;

0-10% of a pigment or dye.

The composition of the invention may advantageously be a liquid two-component curable composition. Further, the composition of the invention may be a non-toxic composition. Additionally, the composition of the invention may have a low solvent content, i.e. less than 5%, in particular less than 2%, more particularly less than 1%, by weight of solvent based on the weight of the composition, or the composition may be substantially free of any solvent.

The composition of the invention is obtained by mixing the prepolymer, the multifunctional resin and the other optional ingredients shortly before use.

The composition of the invention may be used to obtain a sealant, coating or adhesive.

### Sealant, Coating or Adhesive

The sealant, coating or adhesive of the invention is obtained by curing the composition according to the present invention.

The curing may be carried out rapidly under ambient conditions, in the presence of atmospheric moisture. In one embodiment, the curing may be carried out at a temperature of −10 to 50° C., in particular −5 to 45° C., more particularly 0 to 40° C., during a time of 1 to 72 h, in particular 2 to 30 h, more particularly 3 to 24 h.

The sealant, coating or adhesive according to the invention may exhibit a glass transition temperature of −120 to 80° C., preferably −100 to 60° C., more preferably −80 to 50° C.

The sealant, coating or adhesive according to the invention may exhibit excellent mechanical properties. As such, the sealant, coating or adhesive may exhibit a tensile strength at 20° C. of 0.1 to 100 MPa, preferably 1 to 50 MPa, more preferably 5 to 20 MPa. Further, the sealant, coating or adhesive may exhibit an elongation at break at 20° C. of 10 to 1,000%, preferably 50 to 800%, more preferably 100 to 600%.

### Use of the Composition

The invention also relates to the use of the composition according to the invention for producing a sealant, coating or adhesive, especially a leaktight sealant or coating, which has good mechanical strength, is resistant to UV, to oxidation aging, to water and to chemical attack, and which does not have any surface defects or adhesion defects (bubbles, swelling or exudation). The sealants or coatings may be circulable and are particularly suitable for use in an unprotected exterior medium as leaktight sealants. The sealants, coatings or adhesives obtained have an entirely satisfactory water uptake, i.e. less than 8% after 28 days of immersion in water at 20° C. The sealants, coatings or adhesives obtained by the use of the composition according to the invention can cover horizontal, oblique, vertical or rough surfaces and/or surfaces comprising singular points.

The composition of the invention may be used for waterproofing exterior or interior traffic-bearing horizontal surfaces, for making flashings, or for renovating roofs.

In one embodiment, the composition of the invention may be used for waterproofing exterior circulable horizontal surfaces, such as, for example, balconies, stadiums, terraces, car parks, building courtyards, etc.

In another embodiment, the composition of the invention may be used for making upstand flashings, i.e. for making a waterproof coating between a bituminous surface and a vertical wall or a singular point, or alternatively for renovating roofs.

In another embodiment, the composition of the invention may be used to bind two elements together.

The invention will be described in greater detail with the aid of the examples that follow, which are given for purely illustrative purposes.

### EXAMPLES

### Measuring Methods

In the examples, the following methods were used to determine the glass transition temperature (Tg), the ultimate tensile strength, the Young's modulus and the elongation at break.

### Glass Transition Temperature

The glass transition temperature is determined on a dry material at least 7 days after its preparation by differential scan calorimetry (DSC). The DSC analyses were performed on a 10 mg sample using a Q200 apparatus from TA Instruments. The following cycles were applied:

Cycle 1: temperature increase from room temperature to 170° C. at 10° C./min and remaining at 170° C. for 5 min;

Cycle 2: temperature decrease to −80° C. at 20° C./min and remaining at −80° C. for 5 min;

Cycle 3: temperature increase to 170° C. at 10° C./min.

The Tg was measured during the third cycle.

### Mechanical Analysis

The mechanical analyses were determined on a dry material 7 days after its preparation according to standard NF EN ISO 527, February 2012 on an extensometer from Instron. The following parameters were used:

tensile speed: 100 mm/min

temperature: 23° C.

test specimen: dumbbell-shaped type 5.

### Materials

In the examples, the following materials were used:

2-methylpiperazine was obtained from Sigma-Aldrich;

HDDA (1,6-hexanediol diacrylate) was obtained from Sartomer under reference SR238;

ArPUMA (aromatic polyurethane diacrylate) having a number average molecular weight of about 2,500 g·mol⁻¹ was obtained as detailled below;

TMPTA (trimethylolpropane triacrylate) was obtained from Sartomer under reference SR351;

TCDDA (tricyclo[5.2.1.0^{2,6}]decanedimethanol diacrylate) was obtained from Sartomer under reference SR833S;

Calcium carbonate (filler) was obtained from Omya under reference Omya® BLH;

Gray pigment was obtained from Holland Colours under reference Holco XP Dark Grey XP-10-22977.

### Example 1

### Preparation of an Amine Terminated Prepolymer of Formula (I)

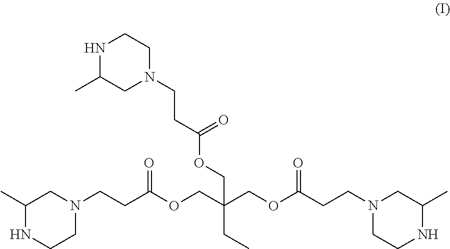

2-methylpiperazine (20.1 g, 0.201 mol) and TMPTA (19.8 g, 0.067 mol) were mixed in a reactor under nitrogen atmosphere, without any catalyst or solvent. The mixture was stirred at 60° C. for 1 hour. The reaction was considered complete when the NMR peaks corresponding to the ethylenic protons «CH₂═CH₂» of the acrylate disappeared (between 5.8 ppm and 6.5 ppm). The resulting product (39.7 g) was a pale yellow viscous liquid. NMR analysis confirmed that the structure of the resulting product corresponded to formula (I). The number average molecular weight was determined by NMR.

NMR-¹H: (δ ppm, CDCl₃) 0.80 (3H), 0.91-0.96 (9H), 1.34-1.60 (5H), 1.66 (3H), 1.94-2.04 (3H), 2.45-2.55 (6H), 2.58-2.68 (6H), 2.69-3.00 (15H), 3.85-4.10 (6H).

The number average molecular weight was determined to be about 596.8 g·mol ⁻¹.

### Example 2

### Preparation of an Amine Terminated Prepolymer of Formula (II)

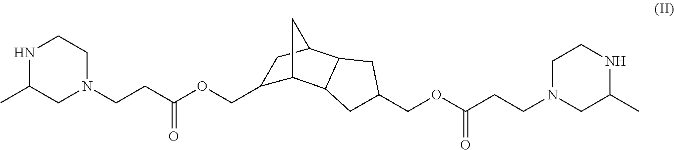

The prepolymer of formula (II) was obtained according to example 1 by reacting 2-methylpiperazine (20.0 g, 0.201 mol) with TCDDA (30.5 g, 0.100 mol) at 60° C. for 1 hour. The resulting product (50.4 g) was a pale yellow viscous liquid. NMR analysis confirmed that the structure of the resulting product corresponded to formula (II).

NMR-¹H: (δ ppm, CDCl₃) 0.80 (3H), 0.91-0.96 (9H), 1.34-1.60 (5H), 1,66 (3H), 1.94-2.04 (3H), 2.45-2.55 (6H), 2.58-2.68 (6H), 2.69-3.00 (15H), 3.85-4.10 (6H).

The number average molecular weight was determined to be about 504.3 g·mol⁻¹.

### Example 3

### Preparation of an Amine Terminated Prepolymer of Formula (III)

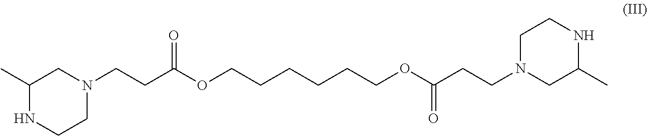

The prepolymer of formula (III) was obtained according to example 1 by reacting 2-methylpiperazine (20.1 g, 0.201 mol) with HDDA (22.6 g, 0.100 mol) at 60° C. for 1 hour. The resulting product (42.6 g) was a pale yellow liquid with low viscosity. NMR analysis confirmed that the structure of the resulting product corresponded to formula (III).

NMR-¹H: (δ ppm, CDCl₃) 0.90-1.010 (6H), 1.30-1.55 (6H), 1.56-1.75 (6H), 2.02 (2H), 2.41-2.55 (4H), 2.60-2.70 (4H), 2.73-3.10 (10H), 4.08 (4H).

The number average molecular weight was determined to be about 426.6 g·mol⁻¹.

### Example 4

### Preparation of an Amine Terminated Prepolymer of Formula (IV)

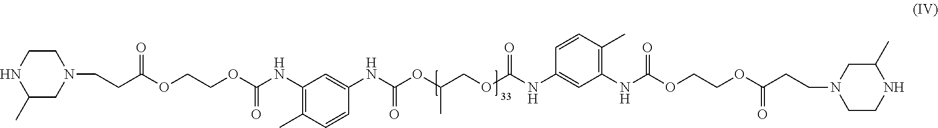

ArPUMA was obtained by reacting poly(propylene glycol) terminated with tolylene 2,4-diisocyanate having a molecular weight of 2,300 g·mol⁻¹ (Sigma-Aldrich) and two equivalents of 2-HEA (2-hydroxyethylacrylate) at a temperature of 70° C. with 0,1% w of DBTDL as catalyst for 1 hour. The end of the reaction by disappearance of NCO peak via FTIR (2273 cm⁻¹).

The prepolymer of formula (IV) was obtained according to example 1 by reacting 2-methylpiperazine (3.2 g, 0.032 mol) with ArPUMA (40 g, 0.016 mol) at 60° C. for 1 hour. The resulting product (43.2 g) was a pale yellow viscous liquid. NMR analysis confirmed that the structure of the resulting product corresponded to formula (IV).

NMR-¹H: (δ ppm, CDCl₃) 0.90-1.40 (118H), 1.85-2.00 (2H), 2.00-2.40 (10H), 2.45-3.10 (18H), 3.25-3.95 (110H), 4.38 (8H), 5.03 (2H), 6.50-7.90 (6H).

The number average molecular weight was determined to be about 2,700 g·mol⁻¹.

### Example 5

### Preparation of Sealant Composition

Compositions 1 to 3 were prepared using the ingredients and the respective amounts in grams listed in the following table:

| | Composition 1 | Composition 2 | Composition 3 |
|---|---|---|---|
| Amine terminated | Prepared in Example 1 | Prepared in Example 2 | Prepared in Example 3 |
| prepolymer | (7.0 g) | (30.3 g) | (30.0 g) |
| Multfunctional resin | ArPUMA | TMPTA | TMPTA |
| | (44.6 g) | (9.6 g) | (11.4 g) |
| | | TCDDA | HDDA |
| | | (3.4 g) | (2.9 g) |
| Filler | Omya ® BLH | Omya ® BLH | Omya ® BLH |
| | (5.9 g) | (5.1 g) | (5.1 g) |
| Pigment | Gray pigment | Gray pigment | Gray pigment |
| | (2.3 g) | (2.1 g) | (2.4 g) |

The amine terminated prepolymer and the multifunctional resin were mixed in a disperser and stirred for 10 minutes. The filler and pigment were then added and the mixture was stirred for 15 minutes. The composition was casted on a plate in order to obtain a uniform film having a thickness of about 1 mm and was left to dry during 7 days.

The thermal and mechanical properties of the resulting sealants are listed in the table below:

| | Composition 1 | Composition 2 | Composition 3 |
|---|---|---|---|
| Ultimate Tensile | 1.12 (+/−0.06) | 2.27 (+/−0.27) | 1.35 (+/−0.18) |
| strength (MPa) | | | |
| Young’s Modulus | 1.42 (+/−0.25) | 1.87 (+/−0.29) | 2.56 (+/−0.07) |
| (MPa) | | | |
| Elongation at break | 380 (+/−30) | 310 (+/−35) | 100 (+/−10) |
| (%) | | | |
| Tg (° C.) | −45 | +17 | −15 |

1. A prepolymer represented by formula (1): 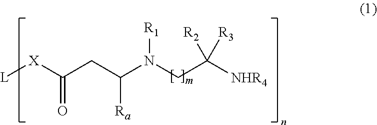
wherein
X is O or NR_{b}, preferably X is O;
L is a plurivalent radical;
Rₐ is hydrogen;
R_{b} is hydrogen;
or Rₐ forms a cycle with R_{b}, preferably a succinimide;
R₁ and R₄ are independently C1-C20 alkyl, C6-C12 aryl or C6-C12 alkylaryl, preferably methyl, ethyl, phenyl or benzyl, more preferably methyl;
or R₁ and R₄ form a cycle, preferably a piperazine, more preferably a piperazine optionally substituted with one or more groups selected from C1-C20 alkyl, C6-C12 aryl, C6-C12 alkylaryl, halogen, C1-C20 haloalkyl, C6-C12 haloaryl and C6-C12 haloalkylaryl, even more preferably a piperazine optionally substituted with one or more groups selected from methyl, ethyl, chloro, phenyl, chlorophenyl and benzyl, more preferably still substituted with methyl;
R₂ and R₃ are independently selected from H, C1-C20 alkyl, C6-C12 aryl, C6-C12 alkylaryl, halogen, C1-C20 haloalkyl, C6-C12 haloaryl or C6-C12 haloalkylaryl, preferably H, methyl, ethyl, chloro, phenyl, chlorophenyl or benzyl, more preferably H or methyl, with the proviso that at least one of R₂ and R₃ is not H;
2≤n≤6, preferably 2≤n≤4, more preferably 2≤n≤3;
m is 1, 2, 3, 4, 5 or 6, preferably m is 1.

2. The prepolymer of claim 1, wherein the prepolymer is represented by one of the following formulae (1a)-(1c): 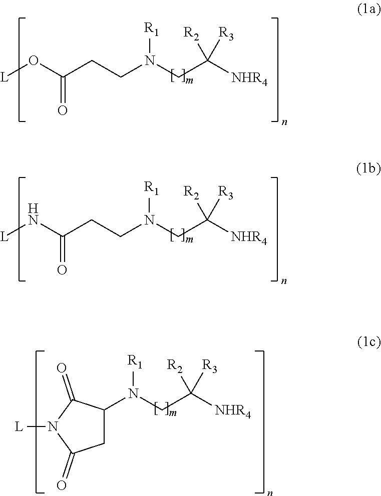
wherein L, R₁, R₂, R₃, R₄, m and n are as defined in claim 1;
preferably the prepolymer is represented by formula (1a).

3. The prepolymer according to claim 1, wherein the following moiety (2) present in formula (1) of the prepolymer: 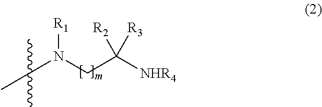
is represented one of the following formulae (2a)-(2b): 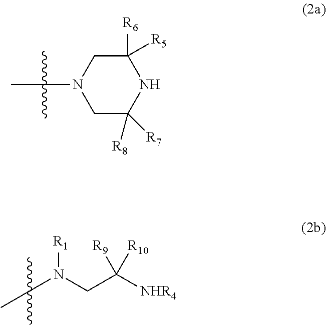
wherein
R₅, R₆, R₇ and R₈ are independently H, C1-C20 alkyl, C6-C12 aryl, C6-C12 alkylaryl, halogen, C1-C20 haloalkyl, C6-C12 haloaryl or C6-C12 haloalkylaryl, preferably H, methyl, ethyl, chloro, phenyl, chlorophenyl or benzyl, more preferably H or methyl; with the proviso that at least one of R₅, R₆, R₇ and R₈ is not H;
R₉ and R₁₀ are independently H, C1-C20 alkyl, C6-C12 aryl, C6-C12 alkylaryl, halogen, C1-C20 haloalkyl, C6-C12 haloaryl or C6-C12 haloalkylaryl, preferably H, methyl, ethyl, chloro, phenyl, chlorophenyl or benzyl, more preferably H or methyl; with the proviso that at least one of R₉ and R₁₀ is not H;
R₁ and R₄ are independently C1-C20 alkyl, C6-C12 aryl or C6-C12 alkylaryl, preferably methyl, ethyl, phenyl or benzyl, more preferably methyl;
preferably moiety (2) is represented by formula (2a) wherein R₅ is C1-C20 alkyl, R₇ is H or C1-C20 alkyl, and R₆ and R₈ are H;
more preferably moiety (2) is represented by formula (2a) wherein R₅ is methyl, R₇ is H or methyl, and R₆ and R₈ are H.

4. The prepolymer according to claim 1, wherein L is selected from a linear or branched, cyclic or acyclic, saturated or unsaturated, aliphatic or aromatic, plurivalent hydrocarbyl radical containing 1 to 500 carbon atoms, said radical being optionally interrupted by one or more functional groups selected from ether (—O—), thioether (—S—), disulfide (—S—S—), ester (—C(O)—O—), amide (—C(O)—NH—), carbamate (—NH—C(O)—O—), urea (—NH—C(O)—NH—), dimethylsiloxane (—Si(Me)₂—O—) and mixtures thereof, said radical optionally having one or more carbon atoms replaced by a nitrogen atom or an isocyanurate group having the following formula: 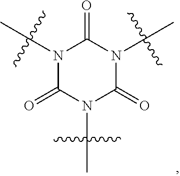 [SMILES: *n1c(=O)n(*)c(=O)n(*)c1=O]
said radical being optionally substituted by one or more substituents selected from halogen, alkyl, aryl, hydroxy (—OH), alkoxy (—OR), haloalkyl, cyano (—CN), carboxyl (—COOH), oxo (═O), formyl (—CHO), ester (—COOR), imido (═NR), amido (—CONHR), a tertiary amino group (—NR₂), nitro (—NO₂), sulfonyl (—SO₂—R) and mixtures thereof, wherein each R is independently C1-C20 alkyl, C6-12 aryl or C6-C12 alkylaryl group.

5. The prepolymer according to claim 1, wherein L is represented by one of the following formulae (La)-(LI3): 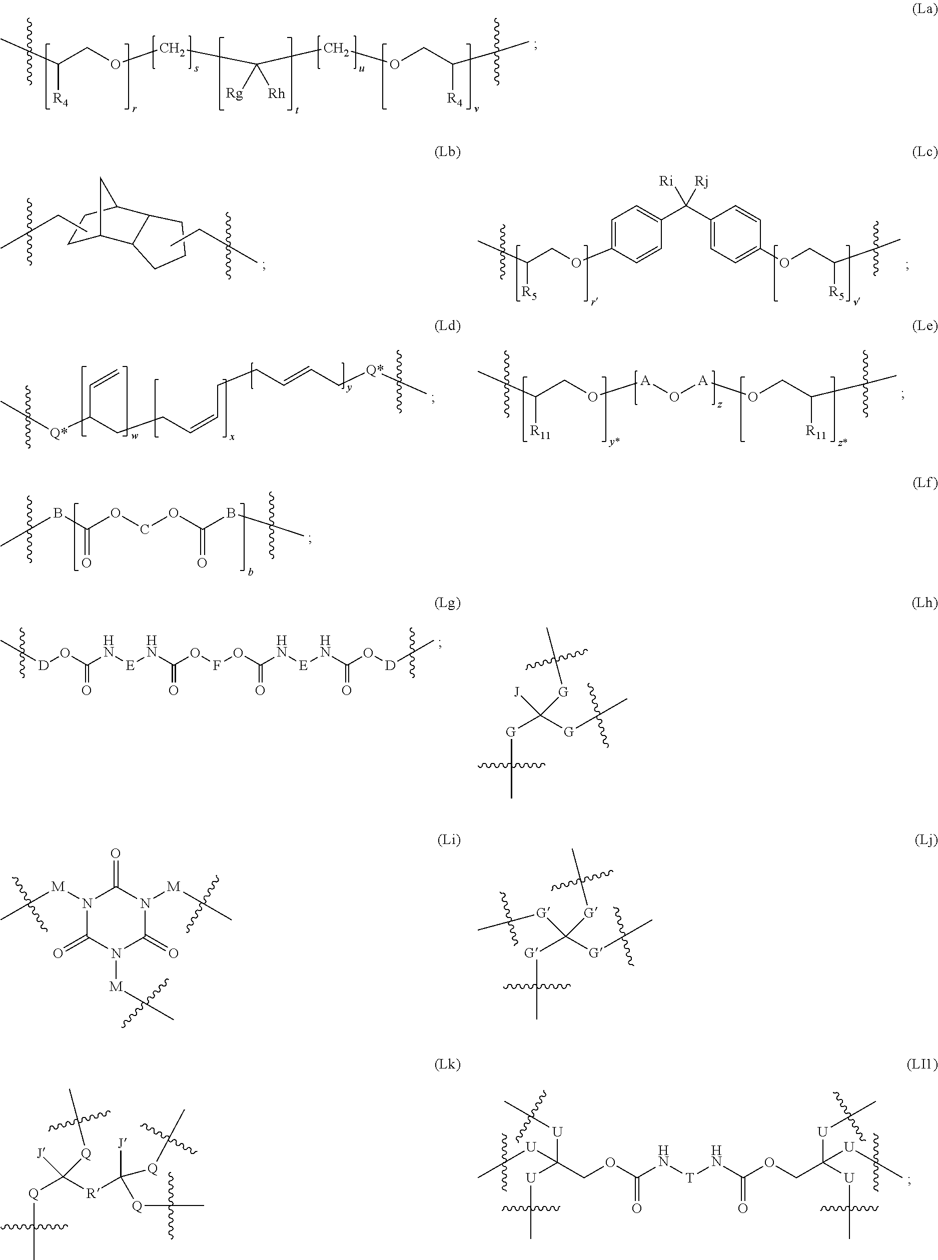 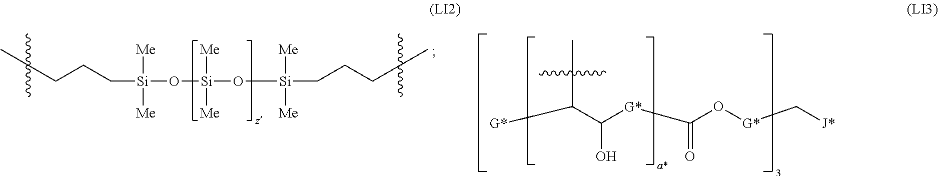
wherein
R_{g} and Rₕ are independently H or C1-C20 alkyl, preferably H, methyl or ethyl, more preferably H or methyl;
Rᵢ and Rⱼ are independently H, halogen, C1-C20 alkyl, C1-C20 haloalkyl, C6-C12 aryl or C6-C12 alkylaryl; preferably C1-C20 alkyl, more preferably methyl;
each R₄, R₅ and R₁₁ is independently H or methyl;
each A is independently a linear or branched, cyclic or acyclic, saturated or unsaturated alkylene comprising 2 to 20 carbon atoms;
each B is independently a linear or branched, cyclic or acyclic, saturated or unsaturated alkylene comprising 2 to 20 carbon atoms;
C is a linear or branched, cyclic or acyclic, saturated or unsaturated alkylene comprising 4 to 100 carbon atoms optionally interrupted by one or more ether and/or ester functional groups;
each D is independently a linear or branched, cyclic or acyclic, saturated or unsaturated alkylene comprising 2 to 20 carbon atoms;
each E is independently a linear or branched, cyclic or acyclic, saturated or unsaturated alkylene comprising 4 to 100 carbon atoms optionally interrupted by one or more ether and/or carbamate functional groups;
each F is independently a linear or branched, cyclic or acyclic, saturated or unsaturated alkylene comprising 4 to 100 carbon atoms optionally interrupted by one or more ether and/or carbamate functional groups;
each G is independently a linear or branched alkylene comprising 0 to 100 carbon atoms optionally interrupted by one or more ether and/or ester functional groups;
each G′ is independently a linear or branched alkylene comprising 0 to 100 carbon atoms optionally interrupted by one or more ether and/or ester functional groups;
each G* is independently a linear or branched alkylene comprising 0 to 100 carbon atoms;
J, J′ and J* are independently H or a linear or branched alkyl comprising 1 to 20 carbon atoms, optionally substituted by hydroxy or alkoxy;
each M is independently a linear or branched, cyclic or acyclic alkylene comprising 1 to 20 carbon atoms optionally interrupted by one or more ether and/or carbamate functional groups;
each Q is independently a linear or branched alkylene comprising 0 to 100 carbon atoms optionally interrupted by one or more ether functional groups;
each Q* is independently a linear or branched alkylene comprising 0 to 100 carbon atoms optionally interrupted by one or more ether and/or ester functional groups;
R′ is a linear or branched alkylene comprising 1 to 20 carbon atoms optionally interrupted by one or more ether functional groups;
T is a linear or branched, cyclic or acyclic, saturated or unsaturated alkylene comprising 4 to 100 carbon atoms optionally interrupted by one or more ether and/or carbamate functional groups;
each U is independently a linear or branched alkylene comprising 0 to 100 carbon atoms optionally interrupted by one or more ether and/or ester functional groups;
b is 1 to 10;
s, t and u are independently 0 to 10;
r, r′, v, v′, w, x, y, y*, z and z* are independently 0 to 50;
z′ is 5 to 150;
each a* is independently 1, 2 or 3 with the proviso that that formula (LI3) does not comprise more than six a* units.

6. The prepolymer according to claim 1, wherein L is represented by the following formula (Lprep) 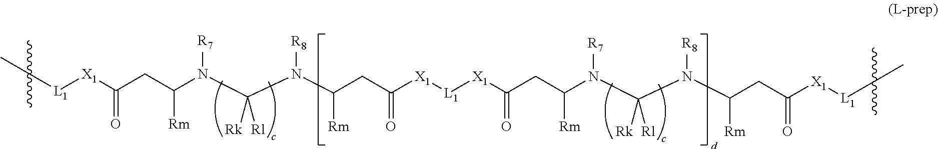
wherein
X₁ is O or NRₙ, preferably X₁ is O;
each L₁ is independently a plurivalent radical, preferably each L₁ having a molecular weight above 500 g·mol⁻¹;
Rₖ is hydrogen;
Rₗ is hydrogen;
or one Rₗ forms a cycle with R₇, another Rₗ forms a cycle with R₈ and the remaining Rₗ are hydrogen, preferably one Rₗ forms a piperidine with R₇, another Rₗ forms a piperidine with R₈ and the remaining Rₗ are hydrogen;
Rₘ is hydrogen;
Rₙ is hydrogen;
or Rₘ forms a cycle with Rₙ, preferably a succinimide;
R₇ and R₈ are independently C1-C20 alkyl, C6-C12 aryl or C6-C12 alkylaryl, preferably methyl, ethyl, phenyl or benzyl, more preferably methyl;
or R₇ and R₈ form a cycle, preferably a piperazine, more preferably a non-substituted piperazine;
or R₇ forms a cycle with one Rₗ and R₈ forms a cycle with another Rₗ, preferably R₇ forms a piperidine with one Rₗ and R₈ forms a piperidine with another Rₗ;
c is 2, 3, 4, 5, 6, 7, 8 or 9;
0≤d≤20, preferably 0.5≤d≤10, more preferably 1≤d≤6, even more preferably 1≤d≤4.

7. A method for preparing a prepolymer, wherein said method comprises reacting an electrophile of formula (4) or (Prep) with a secondary diamine of formula (5): 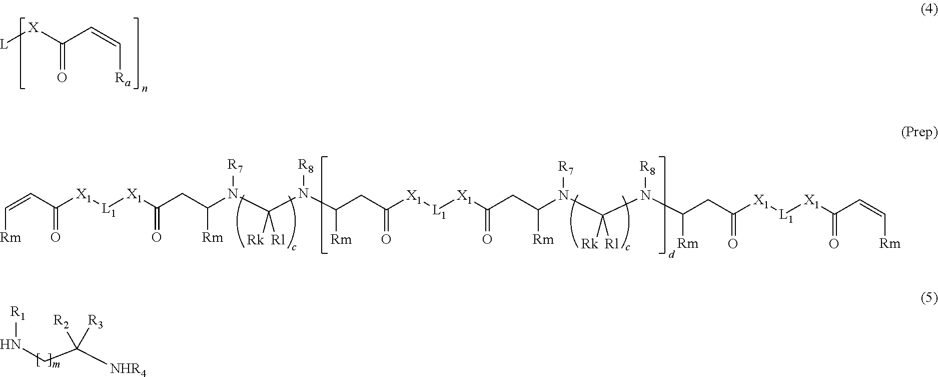
wherein X, L, R₁, R₂, R₃, R₄, Rₐ, m and n are as defined in any one of claims 1 to 5, L₁, X₁, Rₖ, Rₗ, Rₘ, R₇, R₈, c and d are as defined in claim 6;
the molar ratio between the hydrogens on the amine reactive groups of the secondary diamine and the α,β-unsaturated carbonyl groups of the electrophile being from 1.50 to 2.20, preferably 1.80 to 2.10, more preferably 1.90 to 2.00.

8. The method of claim 7, wherein the electrophile is represented by one of the following formulae (4a)-(4c) or (PrepA)-(PrepC): 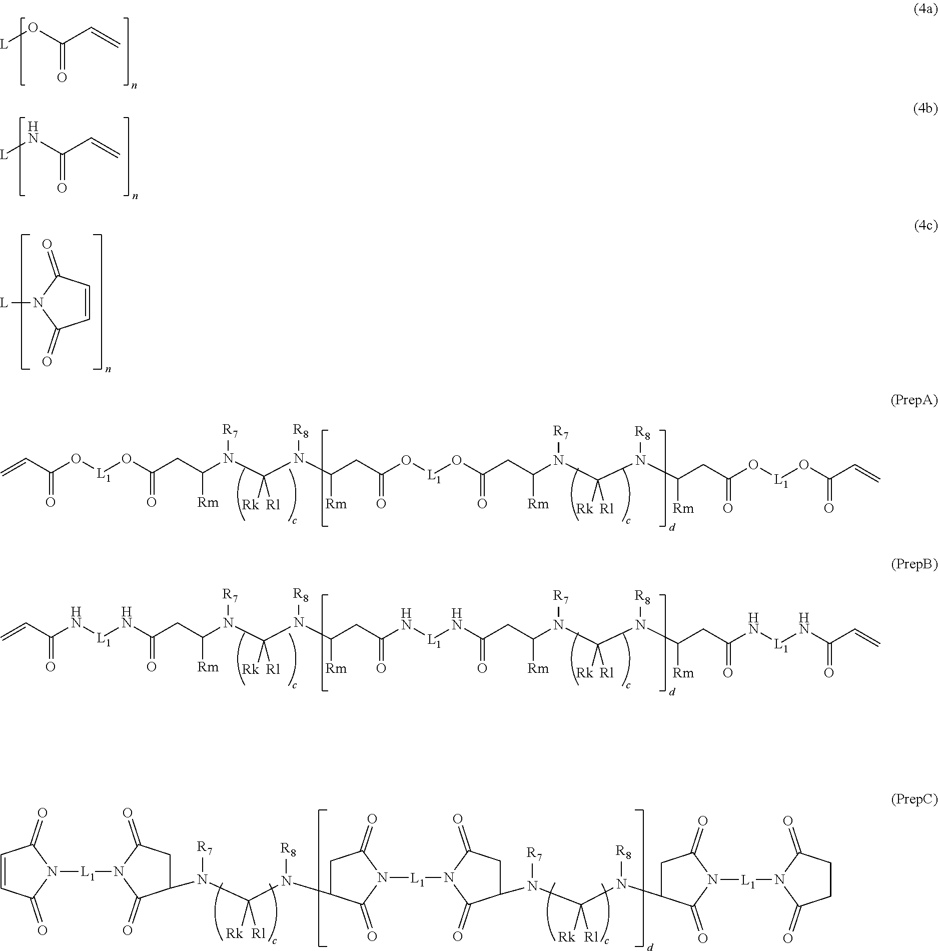
wherein L and n are as defined in any one of claims 1 to 5;
L₁, Rₖ, Rₗ, R₇, R₈, c and d are as defined in claim 6;
preferably the electrophile is represented by formula (4a) or (PrepA).

9. The method of claim 7, wherein the secondary diamine is represented by one of the following formulae (5a) or (5b): 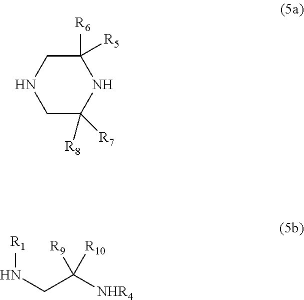
wherein R₁, R₄, R₅, R₆, R₇, R₈, R₉ and R₁₀ are as defined in claim 3;
preferably the secondary diamine is represented by formula (5a) wherein R₅ is C1-C20 alkyl, R₇ is H or C1-C20 alkyl, and R₆ and R₈ are H;
more preferably the secondary diamine is represented by formula (5a) wherein R₅ is methyl, R₇ is H or methyl, and R₆ and R₈ are H.

10. A composition comprising:
a prepolymer as defined in claim 1 or as obtained according to the method of claim 7 or a mixture thereof; and
a multifunctional resin selected from a multifunctional (meth)acrylate resin, a multifunctional epoxy resin, a multifunctional isocyanate resin, a multifunctional carboxylic acid resin, a multifunctional maleimide resin, a multifunctional acrylamide resin, a multifunctional cyclic carbonate resin, an aminoplast resin, and mixtures thereof

11. The composition of claim 10, wherein the multifunctional resin is a multifunctional acrylate resin, preferably a multifunctional polyurethane acrylate resin, a multifunctional polyester acrylate resin (partially or totally bio-based), a bio-based multifunctional epoxide acrylate resin, a multifunctional polyether acrylate, a multifunctional isocyanurate acrylate, a prepolymer of formula (Prep), and mixtures thereof, 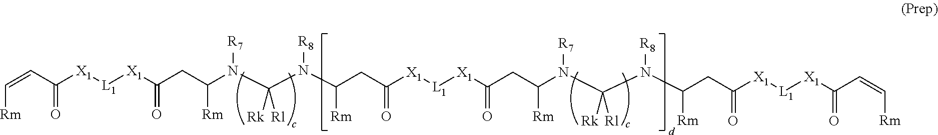
wherein L₁, X₁, Rₖ, Rₗ, Rₘ, R₇, R₈, c et d are as defined in claim 6.

12. The composition of claim 10, comprising a multifunctional resin, preferably a multifunctional acrylate resin, having a functionality higher than 2, preferably from 2.1 to 6, more preferably from 2.5 to 4; and optionally a multifunctional resin, preferably a multifunctional acrylate resin, having a functionality equal to 2; or comprising a multifunctional resin, preferably a multifunctional acrylate resin, having a functionality equal to 2 and optionally a multifunctional resin, preferably a multifunctional acrylate resin, having a functionality higher than 2, preferably from 2.1 to 6, more preferably from 2.5 to 4.

13. The composition of claim 10, wherein the molar ratio between the hydrogens on the amine reactive groups of the prepolymer(s) and the reactive groups of the multifunctional resin is from 0.80 to 1.20, preferably 0.90 to 1.10, more preferably 0.95 to 1.05.

14. The composition of claim 10, wherein the composition further comprises at least one amine terminated prepolymer selected from a secondary amine terminated prepolymer, an aliphatic amine (such as a linear aliphatic amine, cycloaliphatic amine, arylyl amine, polyetheramine), a Mannich base adduct and a polyamine.

15. The composition of claim 10, wherein the composition further comprises a catalyst or a secondary amine terminated prepolymer represented by the following formula (3): 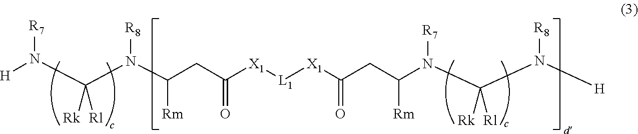
wherein
L₁, X₁, Rₖ, Rₗ, Rₘ, R₇, R₈ and c are as defined in claim 6;
1<d′≤10, preferably 1.5≤d′≤8, more preferably 2≤d′≤6.

16. A sealant, coating or adhesive obtained by curing the composition as defined in claim 10, preferably at a temperature of −10 to 50° C., in particular −5 to 45° C., more particularly 0 to 40° C., during a time of 1 to 72 h, in particular 2 to 30 h, more particularly 3 to 24 h.

17. A method for waterproofing exterior or interior traffic-bearing horizontal surfaces, for making flashings, or for renovating roofs, comprising the step of applying the composition of claim 10.
